(19) United States
(12) Patent Application Publication
(10) Pub. No.: US 2004/0180815 A1
(43) Pub. Date: Sep. 16, 2004

(54) Pyridazinonyl macrocyclic hepatitis C serine protease inhibitors

(76) Inventors: Suanne Nakajima, Cambridge, MA (US); Datong Tang, Malden, MA (US); Frank Wu, Shrewsbury, MA (US); Zhenwei Miao, Medway, MA (US); Ying Sun, Waltham, MA (US); Yat Sun Or, Watertown, MA (US); Zhe Wang, Hockessin, DE (US)

Correspondence Address: ENANTA PHARMACEUTICALS, INC., ATTN: PATENT DEPT., 500 ARSENAL STREET, WATERTOWN, MA, 02472, US

(21) Appl. No.: 10/384,120

(22) Filed: Mar. 7, 2003

(51) Int. Cl. A61K 38/12; C07K 5/12

(52) U.S. Cl. 514/10; 530/317

(57) ABSTRACT

The present invention relates to compounds of Formula I or II, or a pharmaceutically acceptable salt, ester, or prodrug, thereof:

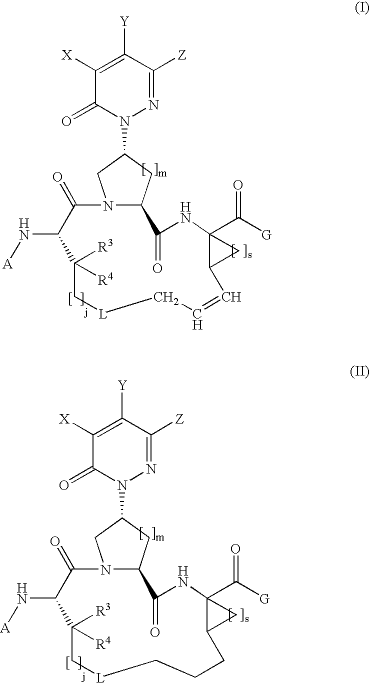

which inhibit serine protease activity, particularly the activity of hepatitis C virus (HCV) NS3-NS4A protease. Consequently, the compounds of the present invention interfere with the life cycle of the hepatitis C virus and are also useful as antiviral agents. The present invention further relates to pharmaceutical compositions comprising the aforementioned compounds for administration to a subject suffering from HCV infection. The invention also relates to methods of treating an HCV infection in a subject by administering a pharmaceutical composition comprising the compounds of the present invention.

### TECHNICAL FIELD

The present invention relates to novel macrocycles having activity against hepatitis C virus (HCV) and useful in the treatment of HCV infections. More particularly, the invention relates to macrocyclic compounds, compositions containing such compounds and methods for using the same, as well as processes for making such compounds.

### BACKGROUND OF THE INVENTION

HCV is the principal cause of non-A, non-B hepatitis and is an increasingly severe public health problem both in the developed and developing world. It is estimated that the virus infects over 200 million people worldwide, surpassing the number of individuals infected with the human immunodeficiency virus (HIV) by nearly five fold. HCV infected patients, due to the high percentage of individuals inflicted with chronic infections, are at an elevated risk of developing cirrhosis of the liver, subsequent hepatocellular carcinoma and terminal liver disease. HCV is the most prevalent cause of hepatocellular cancer and cause of patients requiring liver transplantations in the western world.

There are considerable barriers to the development of anti-HCV therapeutics, which include, but are not limited to, the persistence of the virus, the genetic diversity of the virus during replication in the host, the high incident rate of the virus developing drug-resistant mutants, and the lack of reproducible infectious culture systems and small-animal models for HCV replication and pathogenesis. In a majority of cases, given the mild course of the infection and the complex biology of the liver, careful consideration must be given to antiviral drugs, which are likely to have significant side effects.

Only two approved therapies for HCV infection are currently available. The original treatment regimen generally involves a 3-12 month course of intravenous interferon-α (IFN-α), while a new approved second-generation treatment involves co-treatment with IFN-α and the general antiviral nucleoside mimics like ribavirin. Both of these treatments suffer from interferon related side effects as well as low efficacy against HCV infections. There exists a need for the development of effective antiviral agents for treatment of HCV infection due to the poor tolerability and disappointing efficacy of existing therapies.

In a patient population where the majority of individuals are chronically infected and asymptomatic and the prognoses are unknown, an effective drug must possess significantly fewer side effects than the currently available treatments. The hepatitis C non-structural protein-3 (NS3) is a proteolytic enzyme required for processing of the viral polyprotein and consequently viral replication. Despite the huge number of viral variants associated with HCV infection, the active site of the NS3 protease remains highly conserved thus making its inhibition an attractive mode of intervention. Recent success in the treatment of HIV with protease inhibitors supports the concept that the inhibition of NS3 is a key target in the battle against HCV.

HCV is a flaviridae type RNA virus. The HCV genome is enveloped and contains a single strand RNA molecule composed of circa 9600 base pairs. It encodes a polypeptide comprised of approximately 3010 amino acids.

The HCV polyprotein is processed by viral and host peptidase into 10 discreet peptides which serve a variety of functions. There are three structural proteins, C, E1 and E2. The P7 protein is of unknown function and is comprised of a highly variable sequence. There are six non-structural proteins. NS2 is a zinc-dependent metalloproteinase that functions in conjunction with a portion of the NS3 protein. NS3 incorporates two catalytic functions (separate from its association with NS2): a serine protease at the N-terminal end, which requires NS4A as a cofactor, and an ATP-ase-dependent helicase function at the carboxyl terminus. NS4A is a tightly associated but non-covalent cofactor of the serine protease.

The NS3.4A protease is responsible for cleaving four sites on the viral polyprotein. The NS3-NS4A cleavage is autocatalytic, occurring in cis. The remaining three hydrolyses, NS4A-NS4B, NS4B-NS5A and NS5A-NS5B all occur in trans. NS3 is a serine protease which is structurally classified as a chymotrypsin-like protease. While the NS serine protease possesses proteolytic activity by itself, the HCV protease enzyme is not an efficient enzyme in terms of catalyzing polyprotein cleavage. It has been shown that a central hydrophobic region of the NS4A protein is required for this enhancement. The complex formation of the NS3 protein with NS4A seems necessary to the processing events, enhancing the proteolytic efficacy at all of the sites.

A general strategy for the development of antiviral agents is to inactivate virally encoded enzymes, including NS3, that are essential for the replication of the virus. Current efforts directed toward the discovery of NS3 protease inhibitors were reviewed by S. Tan, A. Pause, Y. Shi, N. Sonenberg, Hepatitis C Therapeutics: Current Status and Emerging Strategies, Nature Rev. Drug Discov., 1, 867-881 (2002). More relevant patent disclosures describing the synthesis of HCV protease inhibitors are: WO 00/59929 (2000); WO 99/07733 (1999); WO 00/09543 (2000); WO 99/50230 (1999); U.S. Pat. No. 5,861,297 (1999).

### SUMMARY OF THE INVENTION

The present invention relates to novel macrocylic compounds and methods of treating a hepatitis C infection in a subject in need of such therapy with said macrocyclic compounds. The present invention further relates to pharmaceutical compositions comprising the compounds of the present invention, or pharmaceutically acceptable salts, esters, or prodrugs thereof, in combination with a pharmaceutically acceptable carrier or excipient.

In one embodiment of the present invention there are disclosed compounds represented by Formulas I and II, or pharmaceutically acceptable salts, esters, or prodrugs thereof:

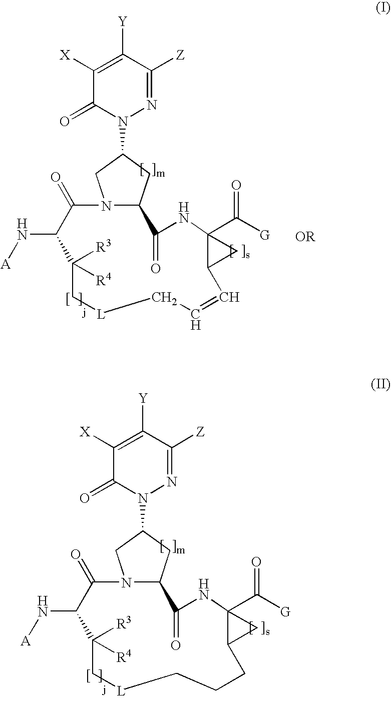

wherein A is H, —(C═O)—R², —(C═O)—O—R¹, —C(═O)—NH—R², —C(═S)—NH—R², or —S(O)₂—R²; G is —OH, —O—(C₁-C₁₂ alkyl), —NHS(O)₂—R¹, —(C═O)—R², —(C═O)—O—R¹, or —(C═O)—NH—R²; L is absent, —S—, —SCH₂—, —SCH₂CH₂—, —S(O)₂—, —S(O)₂CH₂CH₂—, —S(O)—, —S(O)CH₂CH₂—, —O—, —OCH₂—, —OCH₂CH₂—, —(C═O)—CH₂—, —CH(CH₃)CH₂—, —CFHCH₂— or —CF₂CH₂—; X, Y, and Z are independently selected from the group consisting of H, N₃, halogen, C₁-C₆ alkyl, C₃-C₁₂ cycloalkyl, alkylamino, dialkylamino, C₁-C₆ alkynyl, substituted alkynyl, aryl, substituted aryl, —S-aryl, —S-substituted aryl, —O-aryl, —O-substituted aryl, NH-aryl, NH-substituted aryl, diarylamino, diheteroarylamino, arylalkyl, substituted arylalkyl, heteroaryl, substituted heteroaryl, —S-heteroaryl, —S-substituted heteroaryl, —O-heteroaryl, —O-substituted heteroaryl, —NH-heteroaryl, —NH-substituted heteroaryl, heteroarylalkyl, substituted heteroarylalkyl, heterocycloalkyl, and substituted heterocycloalkyl; or, in the alternative, X and Y or Y and Z taken together with the carbon atoms to which they are attached form an aryl, substituted aryl, heteroaryl, or substituted heteroaryl cyclic moiety; j=0, 1, 2, 3, or 4; m=0, 1, or 2; s=0, 1, or 2; R¹ is H, C₁-C₆ alkyl, C₃-C₁₂ cycloalkyl, substituted C₃-C₁₂ cycloalkyl, aryl, substituted aryl, arylalkyl, substituted arylalkyl, heteroaryl, substituted heteroaryl, heteroarylalkyl, substituted heteroarylalkyl, heterocycloalkyl, or substituted heterocycloalkyl; R² is H, C₁-C₆ alkyl, C₃-C₁₂ cycloalkyl, alkylamino, dialkyl amino, arylamino, diarylamino, aryl, substituted aryl, arylalkyl, substituted arylalkyl, heteroaryl, substituted heteroaryl, heteroarylalkyl, substituted heteroarylalkyl, heterocycloalkyl, or substituted heterocycloalkyl; and R³ and R⁴ are each independently hydrogen or methyl.

### DETAILED DESCRIPTION OF THE INVENTION

A first embodiment of the invention is a compound represented by Formula I as described above, or a pharmaceutically acceptable salt, ester or prodrug thereof, in combination with a pharmaceutically acceptable carrier or excipient.

A second embodiment of the invention is a compound represented by Formula II as described above, or a pharmaceutically acceptable salt, ester or prodrug thereof, in combination with a pharmaceutically acceptable carrier or excipient.

Representative subgenera of the invention include, but are not limited to:

A compound of Formula I, wherein A is —(C═O)—O—R¹; G is hydroxyl; L is absent; ; j=3; m=s=1; and R³ and R⁴ are hydrogen;

A compound of Formula I, wherein A is —(C═O)—O-tert-butyl; G is hydroxyl; L is absent; j=3; m=s=1; and R³ and R⁴ are hydrogen;

A compound of Formula II, wherein A is —(C═O)—O—R¹; L is absent; j=3; m=s=1; and R³ and R⁴ are hydrogen; and

A compound of Formula II, wherein A is —(C═O)—O-tert-butyl; G is hydroxyl; L is absent; j=3; m=s=1; and R³ and R⁴ are hydrogen.

Representative compounds of the invention include, but are not limited to, the following compounds:

1. Compound of Formula I, wherein A=tBOC, G=OEt, L=absent, X═Y=bromo, Z=hydrogen, j=3, m=s=1, and R³═R⁴=hydrogen;

2. Compound of Formula I, wherein A=tBOC, G=OEt, L=absent, X═Y=thiophen-3-yl, Z=hydrogen, j=3, m=s=1, and R³═R⁴=hydrogen;

3. Compound of Formula I, wherein A=tBOC, G=OH, L=absent, X═Y=thiophen-3-yl, Z=hydrogen, j=3, m=s=1, and R³═R⁴=hydrogen;

4. Compound of Formula I, wherein A=tBOC, G=OH, L=absent, X═Y=phenyl, Z=hydrogen, j=3, m=s=1, and R³═R⁴=hydrogen;

5. Compound of Formula I, wherein A=tBOC, G=OH, L=absent, X═Y=4-(N,N-dimethylamino)phenyl, Z=hydrogen, j=3, m=s=1, and R³═R⁴=hydrogen;

6. Compound of Formula I, wherein A=tBOC, G=OH, L=absent, X═Y=4-(trifluoromethoxy)phenyl, Z=hydrogen, j=3, m=s=1, and R³═R⁴=hydrogen;

7. Compound of Formula I, wherein A=tBOC, G=OH, L=absent, X═Y=4-(methanesulfonyl)phenyl, Z=hydrogen, j=3, m=s=1, and R³=R 4=hydrogen;

8. Compound of Formula I, wherein A=tBOC, G=OH, L=absent, X═Y=4-(cyano)phenyl, Z=hydrogen, j=3, m=s=1, and R³═R⁴=hydrogen;

9. Compound of Formula I, wherein A=tBOC, G=OH, L=absent, X═Y=3-pyridyl, Z=hydrogen, j=3, m=s=1, and R³═R⁴=hydrogen;

10. Compound of Formula I, wherein A=tBOC, G=OH, L=absent, X═Y=4-(morpholin-4-yl-methanonyl)phenyl, Z=hydrogen, j=3, m=s=1, and R³═R⁴=hydrogen;

11. Compound of Formula I, wherein A=tBOC, G=OH, L=absent, X═Y=bromo, Z=hydrogen, j=3, m=s=1, and R³═R⁴=hydrogen;

12. Compound of Formula I, wherein A=tBOC, G=OH, L=absent, X and Y taken together=phenyl, Z=4-methoxyphenyl, j=3, m=s=1, and R³═R⁴=hydrogen;

13. Compound of Formula I, wherein A=tBOC, G=OH, L=absent, X and Y taken together=phenyl, Z=4-chlorophenyl, j=3, m=s=1, and R³═R⁴=hydrogen;

14. Compound of Formula I, wherein A=tBOC, G=OH, L=absent, X=4-fluorophenyl, Y=hydrogen, Z=phenyl, j=3, m=s=1, and R³═R⁴=hydrogen;

15. Compound of Formula I, wherein A=tBOC, G=OH, L=absent, X=hydrogen, Y=1-piperidyl, Z=phenyl, j=3, m=s=1, and R³═R⁴=hydrogen;

16. Compound of Formula I, wherein A=tBOC, G=OEt, L=absent, X=hydrogen, Y=bromo, Z=phenyl, j=3, m=s=1, and R³═R⁴=hydrogen;

17. Compound of Formula I, wherein A=tBOC, G=OH, L=absent, X=hydrogen, Y=thiophen-3-yl, Z=phenyl, j=3, m=s=1, and R³═R⁴=hydrogen;

18. Compound of Formula I, wherein A=tBOC, G=OEt, L=absent, X=bromo, Y=pyrrolid-1-yl, Z=hydrogen, j=3, m=s=1, and R³═R⁴=hydrogen;

19. Compound of Formula I, wherein A=tBOC, G=OH, L=absent, X=thiophen-3-yl, Y=pyrrolid-1-yl, Z=hydrogen, j=3, m=s=1, and R³═R⁴=hydrogen;

20. Compound of Formula I, wherein A=tBOC, G=OEt, L=absent, X=bromo, Y=azido, Z=hydrogen, j=3, m=s=1, and R³═R⁴=hydrogen;

21. Compound of Formula I, wherein A=tBOC, G=OEt, L=absent, X=thiophen-3-yl, Y=azido, Z=hydrogen, j=3, m=s=1, and R³═R⁴=hydrogen;

22. Compound of Formula I, wherein A=tBOC, G=OH, L=absent, X=thiophen-3-yl, Y=azido, Z=hydrogen, j=3, m=s=1, and R³═R⁴=hydrogen;

23. Compound of Formula I, wherein A=tBOC, G=OH, L=absent, X=thiophen-3-yl, Y=tetrazol-2-yl, Z=hydrogen, j=3, m=s=1, and R³═R⁴=hydrogen;

24. Compound of Formula I, wherein A=tBOC, G=OH, L=absent, X═Y=mercapto-2-pryrimidine, Z=hydrogen, j=3, m=s=1, and R³═R⁴=hydrogen;

25. Compound of Formula I, wherein A=tBOC, G=OH, L=absent, X=bromo, Y=mercapto-2-pryrimidine, Z=hydrogen, j=3, m=s=1, and R³═R⁴=hydrogen;

26. Compound of Formula I, wherein A=tBOC, G=OH, L=absent, X=thiophen-3-yl, Y=mercapto-2-pryrimidine, Z=hydrogen, j=3, m=s=1, and R³═R⁴=hydrogen;

27. Compound of Formula I, wherein A=tBOC, G=OH, L=absent, X═Y=thiazol-2-yl, Z=hydrogen, j=3, m=s=1, and R³═R⁴=hydrogen;

28. Compound of Formula I, wherein A=tBOC, G=OH, L=absent, X═Y=imidazol-1-yl, Z=hydrogen, j=3, m=s=1, and R³═R⁴=hydrogen;

29. Compound of Formula I, wherein A=tBOC, G=OH, L=absent, X=2-(cyclopropylamino)-thiazol-4-yl, Y=4-methoxyphenyl, Z=hydrogen, j=3, m=s=1, and R³═R⁴=hydrogen;

30. Compound of Formula I, wherein A=tBOC, G=OH, L=absent, X and Y taken together=6-methoxy-isoquinolinyl, Z=hydrogen, j=3, m=s=1, and R³═R⁴=hydrogen;

31. Compound of Formula I, wherein A=—(C═O)—O—R¹, wherein R¹=cyclopentyl, G=OH, L=absent, X=thiophen-3-yl, Y=thiophen-3-yl, Z=hydrogen, j=3, m=s=1, and R³═R⁴=hydrogen;

32. Compound of Formula I, wherein A=—(C═O)—O—R¹, wherein R¹=cyclobutyl, G=OH, L=absent, X=thiophen-3-yl, Y=thiophen-3-yl, Z=hydrogen, j=3, m=s=1, and R³═R⁴=hydrogen;

33. Compound of Formula I, wherein A=—(C═O)—O—R¹, wherein R¹=cyclohexyl, G=OH, L=absent, X=thiophen-3-yl, Y=thiophen-3-yl, Z=hydrogen, j=3, m=s=1, and R³═R⁴=hydrogen;

34. Compound of Formula I, wherein A=—(C═O)—O—R¹, wherein R¹=

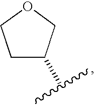

G=OH, L=absent X=thiophen-3-yl, Y=thiophen-3-yl, Z=hydrogen, j=3, m=s=1, and R³═R⁴=hydrogen;

35. Compound of Formula I, wherein A=—(C═O)—O—R¹, wherein R¹=

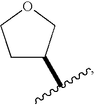

G=OH, L=absent, X=thiophen-3-yl, Y=thiophen-3-yl, Z=hydrogen, j=3, m=s=1, and R³═R⁴=hydrogen; and

36. Compound of Formula I, wherein A=—(C═O)—O—R¹, wherein R¹=

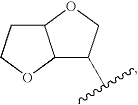

G=OH, L=absent, X=thiophen-3-yl, Y=thiophen-3-yl, Z=hydrogen, j=3, m=s=1, and R³═R⁴=hydrogen;

37. Compound of Formula I, wherein A=tBOC, G=OH, L=—(C═O)CH₂—, X=thiophen-3-yl, Y=thiophen-3-yl, Z=hydrogen, j=1, m=s=1, and R³═R⁴=hydrogen;

38. Compound of Formula I, wherein A=tBOC, G=OH, L=—CH(CH₃)CH₂—, X=thiophen-3-yl, Y=thiophen-3-yl, Z=hydrogen, j=1, m=s=1, R³=methyl, and R⁴=hydrogen;

39. Compound of Formula I, wherein A=tBOC, G=OH, L=—O—, X=thiophen-3-yl, Y=thiophen-3-yl, Z=hydrogen, j=0, m=s=1, R³=methyl, and R⁴=hydrogen;

40. Compound of Formula I, wherein A=tBOC, G=OH, L=—S—, X=thiophen-3-yl, Y=thiophen-3-yl, Z=hydrogen, j=0, m=s=1, R³=methyl, and R⁴=hydrogen;

41. Compound of Formula I, wherein A=tBOC, G=OH, L=—S(O)—, X=thiophen-3-yl, Y=thiophen-3-yl, Z=hydrogen, j=2, m=s=1, R³=methyl, and R⁴=hydrogen;

42. Compound of Formula I, wherein A=tBOC, G=OH, L=—S(O)₂—, X=thiophen-3-yl, Y=thiophen-3-yl, Z=hydrogen, j=2, m=s=1, R³=methyl, and R⁴=hydrogen;

43. Compound of Formula I, wherein A=tBOC, G=OH, L=—SCH₂CH₂—, X=thiophen-3-yl, Y=thiophen-3-yl, Z=hydrogen, j=0, m=s=1, and R³═R⁴=CH₃;

44. Compound of Formula I, wherein A=tBOC, G=OH, L=—CF₂CH₂—, X=thiophen-3-yl, Y=thiophen-3-yl, Z=hydrogen, j=1, m=s=1, and R³═R⁴=hydrogen;

45. Compound of Formula I, wherein A=tBOC, G=OH, L=—CFHCH₂—, X=thiophen-3-yl, Y=thiophen-3-yl, Z=hydrogen, j=1, m=s=1, and R³═R⁴=hydrogen;

46. Compound of Formula I, wherein A=—(C═O)—O—R¹, R¹=cyclopentyl, G=—O-phenethyl, L=absent, X=thiophen-3-yl, Y=thiophen-3-yl, Z=hydrogen, j=3, m=s=1, and R³═R⁴=hydrogen;

47. Compound of Formula I, wherein A=—(C═O)—O—R¹, R¹=cyclopentyl, G=—NH-phenethyl, L=absent, X=thiophen-3-yl, Y=thiophen-3-yl, Z=hydrogen, j=3, m=s=1, and R³═R⁴=hydrogen;

48. Compound of Formula I, wherein A=—(C═O)—O—R¹, R¹=cyclopentyl, G=—NHS(O)₂-phenethyl, L=absent, X=thiophen-3-yl, Y=thiophen-3-yl, Z=hydrogen, j=3, m=s=1, and R³═R⁴=hydrogen;

49. Compound of Formula I, wherein A=—(C═O)—O—R¹, R¹=cyclopentyl, G=—(C═O)—OH, L=absent, X=thiophen-3-yl, Y=thiophen-3-yl, Z=hydrogen, j=3, m=s=1, and R³═R⁴=hydrogen;

50. Compound of Formula I, wherein A=—(C═O)—O—R¹, R¹=cyclopentyl, G=—(C═O)—O-phenethyl, L=absent, X=thiophen-3-yl, Y=thiophen-3-yl, Z=hydrogen, j=3, m=s=1, and R³═R⁴=hydrogen;

51. Compound of Formula I, wherein A=—(C═O)—O—R¹, R¹=cyclopentyl, G=—(C═O)—NH-phenethyl, L=absent, X=thiophen-3-yl, Y=thiophen-3-yl, Z=hydrogen, j=3, m=s=1, and R³═R⁴=hydrogen; and

52. Compound of Formula I, wherein A=—(C═O)—O—R¹, R¹=cyclopentyl, G=—(C═O)—NH—S(O)₂-benzyl, L=absent, X=thiophen-3-yl, Y=thiophen-3-yl, Z=hydrogen, j=3, m=s=1, and R³═R⁴=hydrogen.

According to an alternate embodiment, the pharmaceutical compositions of the present invention may further contain other anti-HCV agents. Examples of anti-HCV agents include, but are not limited to, α-interferon, β-interferon, ribavirin, and amantadine.

According to an additional alternate embodiment, the pharmaceutical compositions of the present invention may further contain other HCV protease inhibitors.

According to yet another alternate embodiment, the pharmaceutical compositions of the present invention may further comprise inhibitor(s) of other targets in the HCV life cycle, including, but not limited to, helicase, polymerase, metalloprotease, and internal ribosome entry site (IRES).

According to a further embodiment, the present invention includes methods of treating hepatitis C infections in a subject in need of such treatment by administering to said subject an anti-HCV virally effective amount of the pharmaceutical compositions of the present invention.

Definitions

Listed below are definitions of various terms used to describe this invention. These definitions apply to the terms as they are used throughout this specification and claims, unless otherwise limited in specific instances, either individually or as part of a larger group.

The terms “C₁-C₃ alkyl,” “C₁-C₆ alkyl” or “C₁-C₁₂ alkyl,” as used herein, refer to saturated, straight- or branched-chain hydrocarbon radicals containing between one and three, one and twelve, or one and six carbon atoms, respectively. Examples of C₁-C₃ alkyl radicals include methyl, ethyl, propyl and isopropyl radicals; examples of C₁-C₆ alkyl radicals include, but are not limited to, methyl, ethyl, propyl, isopropyl, n-butyl, tert-butyl, neopentyl and n-hexyl radicals; and examples of C₁-C₁₂ alkyl radicals include, but are not limited to, ethyl, propyl, isopropyl, n-hexyl, octyl, decyl, dodecyl radicals.

The term “substituted alkyl,” as used herein, refers to a “C₂-C₁₂ alkyl” or “C₁-C₆ alkyl” group substituted by independent replacement of one, two or three of the hydrogen atoms thereon with F, Cl, Br, I, OH, NO₂, CN, C₁-C₆-alkyl-OH, C(O)—C₁-C₆-alkyl, OCH₂—C₃—C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The terms “C₂-C₁₂ alkenyl” or “C₁-C₆ alkenyl,” as used herein, denote a monovalent group derived from a hydrocarbon moiety containing from two to twelve or two to six carbon atoms having at least one carbon-carbon double bond by the removal of a single hydrogen atom. Alkenyl groups include, but are not limited to, for example, ethenyl, propenyl, butenyl, 1-methyl-2-buten-1-yl, and the like.

The term “substituted alkenyl,” as used herein, refers to a “C₂-C₁₂ alkenyl” or “C₁-C₆ alkenyl” group substituted by independent replacement of one, two or three of the hydrogen atoms thereon with F, Cl, Br, I, OH, NO₂, CN, C₁-C₆-alkyl-OH, C(O)—C₁-C₆-alkyl, OCH₂—C₃-C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The terms “C₁-C₁₂ alkynyl” or “C₁-C₆ alkynyl,” as used herein, denote a monovalent group derived from a hydrocarbon moiety containing from two to twelve or two to six carbon atoms having at least one carbon-carbon triple bond by the removal of a single hydrogen atom. Representative alkynyl groups include, but are not limited to, for example, ethynyl, 1-propynyl, 1-butynyl, and the like.

The term “substituted alkynyl,” as used herein, refers to a “C₂-C₁₂ alkynyl” or “C₁-C₆ alkynyl” group substituted by independent replacement of one, two or three of the hydrogen atoms thereon with F, Cl, Br, I, OH, NO₂, CN, C₁-C₆-alkyl-OH, C(O)—C₁-C₆-alkyl, OCH₂—C₃-C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂—C₁-C₆-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The term “C₁-C₆ alkoxy,” as used herein, refers to a C₁-C₆ alkyl group, as previously defined, attached to the parent molecular moiety through an oxygen atom. Examples of C₁-C₆-alkoxy include, but are not limited to, methoxy, ethoxy, propoxy, isopropoxy, n-butoxy, tert-butoxy, neopentoxy and n-hexoxy.

The terms “halo” and “halogen,” as used herein, refer to an atom selected from fluorine, chlorine, bromine and iodine.

The term “aryl,” as used herein, refers to a mono- or bicyclic carbocyclic ring system having one or two aromatic rings including, but not limited to, phenyl, naphthyl, tetrahydronaphthyl, indanyl, idenyl and the like.

The term “substituted aryl,” as used herein, refers to an aryl group, as defined herein, substituted by independent replacement of one, two or three of the hydrogen atoms thereon with F, Cl, Br, I, OH, NO₂, CN, C₁-C₆-alkyl-OH, C(O)—C₁-C₆-alkyl, OCH₂—C₃-C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The term “arylalkyl,” as used herein, refers to a C₁-C₃ alkyl or C₁-C₆ alkyl residue attached to an aryl ring. Examples include, but are not limited to, benzyl, phenethyl and the like.

The term “substituted arylalkyl,” as used herein, refers to an arylalkyl group, as previously defined, substituted by independent replacement of one, two or three of the hydrogen atoms thereon with F, Cl, Br, I, OH, NO₂, CN, C₁-C₆-alkyl-OH, C(O)—C₁-C₆-alkyl, OCH₂—C₃-C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The term “heteroaryl,” as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

The term “substituted heteroaryl,” as used herein, refers to a heteroaryl group as defined herein, substituted by independent replacement of one, two or three of the hydrogen atoms thereon with F, Cl, Br, I, OH, NO₂, CN, C₁-C₆-alkyl-OH, C(O)—C₁-C₆-alkyl, OCH₂—C₃-C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The term “C₃-C₁₂-cycloalkyl” denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo [2.2.1] heptyl, and bicyclo [2.2.2] octyl.

The term “substituted C₃-C₁₂-cycloalkyl,” as used herein, refers to a C₃-C₁₂-cycloalkyl group as defined herein, substituted by independent replacement of one, two or three of the hydrogen atoms thereon with F, Cl, Br, I, OH, NO₂, CN, C₁-C₆-alkyl-OH, C(O)—C₁-C₆-alkyl, OCH₂—C₃-C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The term “heterocycloalkyl,” as used herein, refers to a non-aromatic 5-, 6- or 7-membered ring or a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “substituted heterocycloalkyl,” as used herein, refers to a heterocycloalkyl group, as previously defined, substituted by independent replacement or one, two, or three of the hydrogen atoms thereon with F, Cl, Br, I, OH, NO₂, CN, C₁-C₆-alkyl-OH, C(O)—C₁-C₆-alkyl, OCH₂—C₃-C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂-C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The term “heteroarylalkyl,” as used herein, refers to a C₁-C₃ alkyl or C₁-C₆ alkyl residue residue attached to a heteroaryl ring. Examples include, but are not limited to, pyridinylmethyl, pyrimidinylethyl and the like.

The term “substituted heteroarylalkyl,” as used herein, refers to a heteroarylalkyl group, as previously defined, substituted by independent replacement or one, two, or three of the hydrogen atoms thereon with F, Cl, Br, I, OH, NO₂, CN, C₁-C₆-alkyl-OH, C(O)—C₁-C₆-alkyl, OCH₂—C₃-C₁₂-cycloalkyl, C(O)-aryl, C(O)-heteroaryl, CO₂-alkyl, CO₂-aryl, CO₂-heteroaryl, CONH₂, CONH—C₁-C₆-alkyl, CONH-aryl, CONH-heteroaryl, OC(O)—C₁-C₆-alkyl, OC(O)-aryl, OC(O)-heteroaryl, OCO₂-alkyl, OCO₂-aryl, OCO₂-heteroaryl, OCONH₂, OCONH—C₁-C₆-alkyl, OCONH-aryl, OCONH-heteroaryl, NHC(O)—C₁-C₆-alkyl, NHC(O)-aryl, NHC(O)-heteroaryl, NHCO₂-alkyl, NHCO₂-aryl, NHCO₂-heteroaryl, NHCONH₂, NHCONH—C₁-C₆-alkyl, NHCONH-aryl, NHCONH-heteroaryl, SO₂—C₁-C₆-alkyl, SO₂-aryl, SO₂-heteroaryl, SO₂NH₂, SO₂NH—C₁-C₆-alkyl, SO₂NH-aryl, SO₂NH-heteroaryl, C₁-C₆-alkyl, C₃-C₆-cycloalkyl, CF₃, CH₂CF₃, CHCl₂, CH₂NH₂, CH₂SO₂CH₃H, C₁-C₆ alkyl, halo alkyl, C₃-C₁₂ cycloalkyl, substituted C₃-C₁₂ cycloalkyl, aryl, substituted aryl, arylalkyl, heteroaryl, heteroarylalkyl, heterocycloalkyl, benzyl, benzyloxy, aryloxy, heteroaryloxy, C₁-C₆-alkoxy, methoxymethoxy, methoxyethoxy, amino, benzylamino, arylamino, heteroarylamino, C₁-C₃-alkylamino, thio, aryl-thio, heteroarylthio, benzyl-thio, C₁-C₆-alkyl-thio, or methylthiomethyl.

The term “alkylamino” refers to a group having the structure —NH(C₁-C₁₂ alkyl) where C₁-C₁₂ alkyl is as previously defined.

The term “dialkylamino” refers to a group having the structure —N(C₁-C₁₂ alkyl)₂ where C₁-C₁₂ alkyl is as previously defined. Examples of dialkylamino are, but not limited to, N,N-dimethylamino, N,N-diethylamino, N,N-methylethylamino, piperidino, and the like.

The term “diarylamino” refers to a group having the structure —N(aryl)₂ or —N(substituted aryl)₂ where substituted aryl is as previously defined. Examples of diarylamino are, but not limited to, N,N- diphenylamino, N,N-dinapthylamino, N,N-di(toluenyl)amino, and the like.

The term “diheteroarylamino” refers to a group having the structure —N(heteroaryl)₂ or —N(substituted heteroaryl)₂, where heteroaryl and substituted heteroaryl is as previously defined. Examples of diheteroarylamino are, but not limited to, N,N-difuranylamino, N,N-dithiazolidinylamino, N,N-di(imidazole)amino, and the like.

The compounds described herein contain two or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion.

The term “subject” as used herein refers to a mammal. Preferably the mammal is a human. A subject also refers to, for example, dogs, cats, horses, cows, pigs, guinea pigs and the like.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. The salts can be prepared in situ during the final isolation and purification of the compounds of the invention, or separately by reacting the free base function with a suitable organic acid. Examples of pharmaceutically acceptable, nontoxic acid addition salts include, but are not limited to, salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, C₁-C₆ sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, but are not limited to, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/reward ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design (American Pharmaceutical Association and Pergamon Press, 1987), both of which are incorporated by reference herein.

The compounds of this invention may be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Pharmaceutical Compositions

The pharmaceutical compositions of the present invention comprise a therapeutically effective amount of a compound of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some Examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Antiviral Activity

According to the methods of treatment of the present invention, viral infections are treated or prevented in a subject such as a human or lower mammal by administering to the subject a therapeutically effective amount of a compound of the invention, in such amounts and for such time as is necessary to achieve the desired result. The term “anti-hepatitis C virally effective amount” of a compound of the invention, as used herein, means a sufficient amount of the compound so as to decrease the viral load in a subject, thus decreasing said subject's chronic HCV symptoms. As well understood in the medical arts an anti-hepatitis C virally effective amount of a compound of this invention will be at a reasonable benefit/risk ratio applicable to any medical treatment.

Upon improvement of a subject's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present invention will be decided by the attending physician within the scope of sound medical judgment. The specific anti-HCV virally effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily dose of the compounds of this invention administered to a subject in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

Unless otherwise defined, all technical and scientific terms used herein are accorded the meaning commonly known to one with ordinary skill in the art. All publications, patents, published patent applications, and other references mentioned herein are hereby incorporated by reference in their entirety.

Abbreviations

Abbreviations which have been used in the descriptions of the schemes and the examples that follow are:

ACN for acetonitrile;

BME for 2-mercaptoethanol;

BOP for benzotriazol-1-yloxy-tris(dimethylamino)phosphonium hexafluorophosphate;

COD for cyclooctadiene;

DAST for diethylaminosulfur trifluoride;

DABCYL for 6-(N-4′-carboxy-4-(dimethylamino)azobenzene)-aminohexyl-1-O-(2-cyanoethyl)-(N,N-diisopropyl)-phosphoramidite;

DCM for dichloromethane;

DIAD for diisopropyl azodicarboxylate;

DIBAL-H for diisobutylaluminum hydride;

DIEA for diisopropyl ethylamine;

DMAP for N,N-dimethylaminopyridine;

DME for ethylene glycol dimethyl ether;

DMEM for Dulbecco's Modified Eagles Media;

DMF for N,N-dimethyl formamide;

DMSO for dimethylsulfoxide;

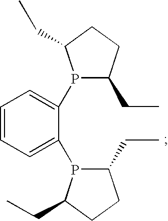

DUPHOS for

EDANS for 5-(2-Amino-ethylamino)-naphthalene-1-sulfonic acid;

EDCI or EDC for 1-(3-diethylaminopropyl)-3-ethylcarbodiimide hydrochloride;

EtOAc for ethyl acetate;

HATU for O (7-Azabenzotriazole-1-yl)-N,N,N′,N′-tetramethyluronium hexafluorophosphate;

Hoveyda's Cat. for Dichloro(o-isopropoxyphenylmethylene) (tricyclohexylphosphine)ruthenium(II);

KHMDS is potassium bis(trimethylsilyl) amide;

Ms for mesyl;

NMM for N-4-methylmorpholine

PyBrOP for Bromo-tri-pyrolidino-phosphonium hexafluorophosphate;

Ph for phenyl;

RCM for ring-closing metathesis;

RT for room temperature;

RT-PCR for reverse transcription-polymerase chain reaction;

TEA for triethyl amine;

TFA for trifluoroacetic acid;

THF for tetrahydrofuran;

TLC for thin layer chromatography;

TPP or PPh₃ for triphenylphosphine;

tBOC or Boc for tert-butyloxy carbonyl; and

Xantphos for 4,5-Bis-diphenylphosphanyl-9,9-dimethyl-9H-xanthene.

Synthetic Methods

The compounds and processes of the present invention will be better understood in connection with the following synthetic schemes which illustrate the methods by which the compounds of the invention may be prepared.

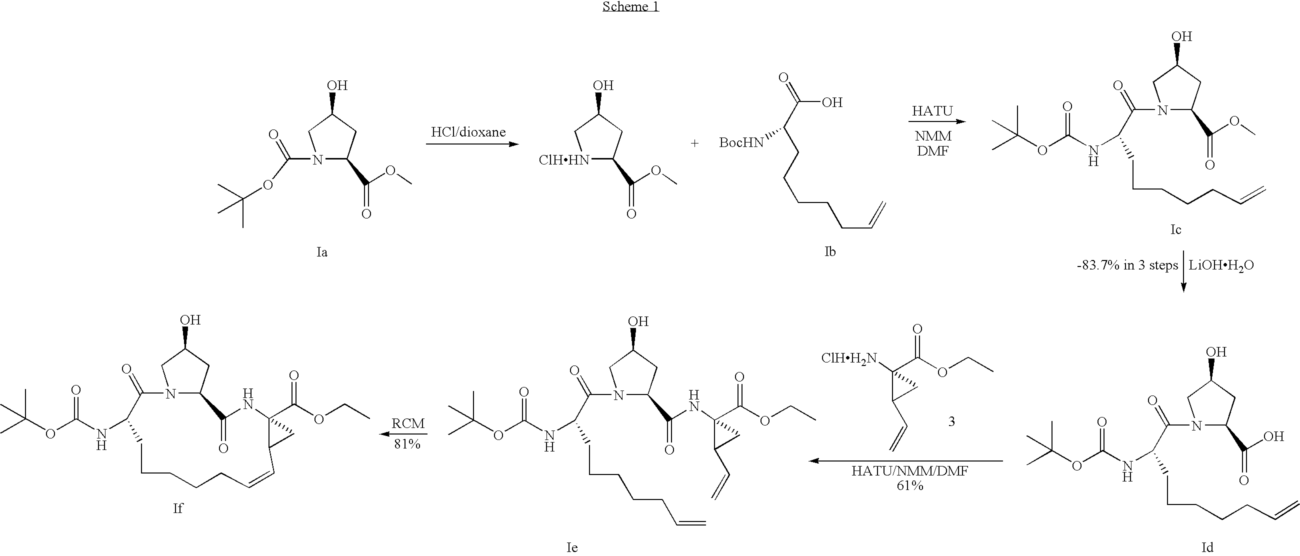

All of the pyridazinone analogs were prepared from the common intermediate If. The synthesis of compound If is outlined in Scheme 1. Commercially available boc-hydroxyproline Ia is treated with HCl in dioxane and is further coupled with acid Ib using HATU to afford intermediate Ic. Hydrolysis of Ic with LiOH followed by another peptide coupling with cyclopropyl amine Id yielded the tri-peptide Ie. Finally, ring closure methathesis with Hoveyda's 1^{st} generation catalyst gave the desired key intermediate If.

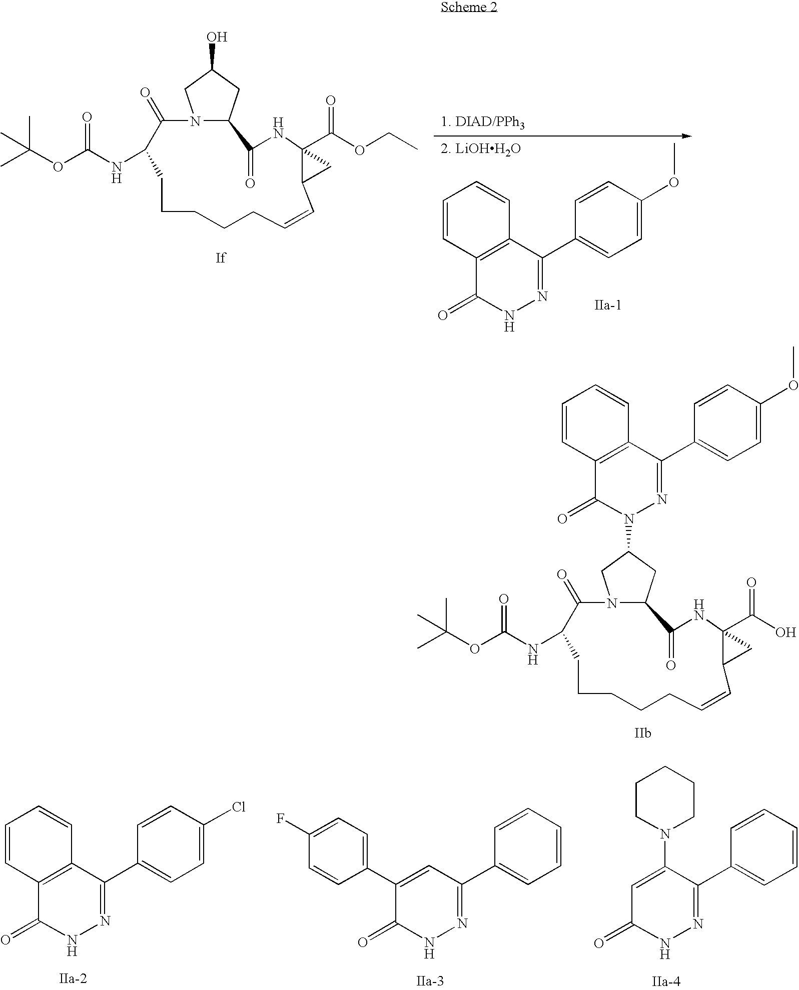

The pyridazinone analogs used in the present invention were prepared via several different synthetic routes. The simplest method, shown in Scheme 2, is to condense commercially available pyridazinones (IIa-1-IIa-4) with key intermediate If by using Mitsunobu conditions followed by hydrolysis with LiOH. For further details on the Mitsunobu reaction see O. Mitsunobu, Synthesis 1981, 1-28; D. L. Hughes, Org. React. 29, 1-162 (1983); D. L. Hughes, Organic Preparations and Procedures Int. 28, 127-164 (1996); and J. A. Dodge, S. A. Jones, Recent Res. Dev. Org. Chem. 1, 273-283 (1997).

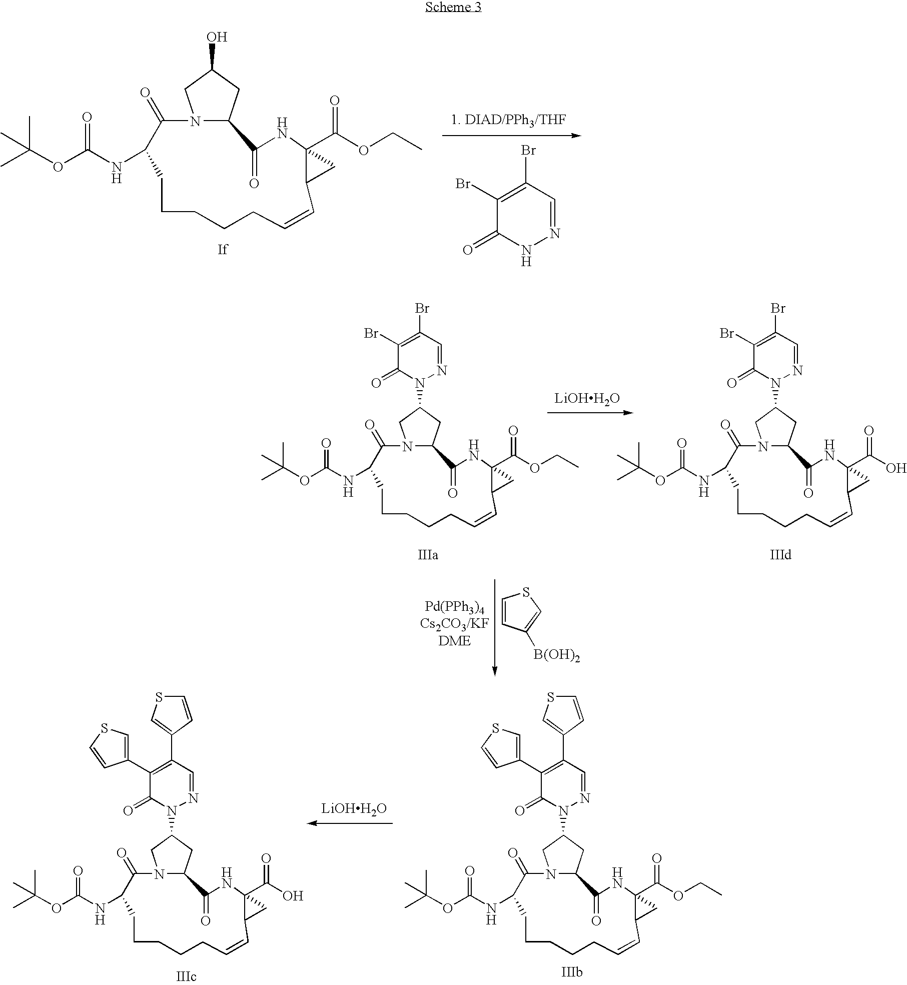

The second method of preparing pyridazinone analogs of the present invention is to further chemically manipulate di-bromo intermediate IIIa (Scheme 3). The standard Mitsunobu coupling of the commercially available 4,5-dibromopyridazinone with hydroxyl If afforded the desired macrocycle IIIa. Coupling of IIIa with excess 3-thiophene boronic acid, cesium carbonate and potassium fluoride furnished di-thiophene IIIb. Hydrolysis of compound compounds IIIa and IIIb with LiOH gave the desired analogs IIId and IIIc respectively. Many different boronic acids may be used in a similar manner to yield a plethora of di-substituted pyridazinonyl macrocycles.

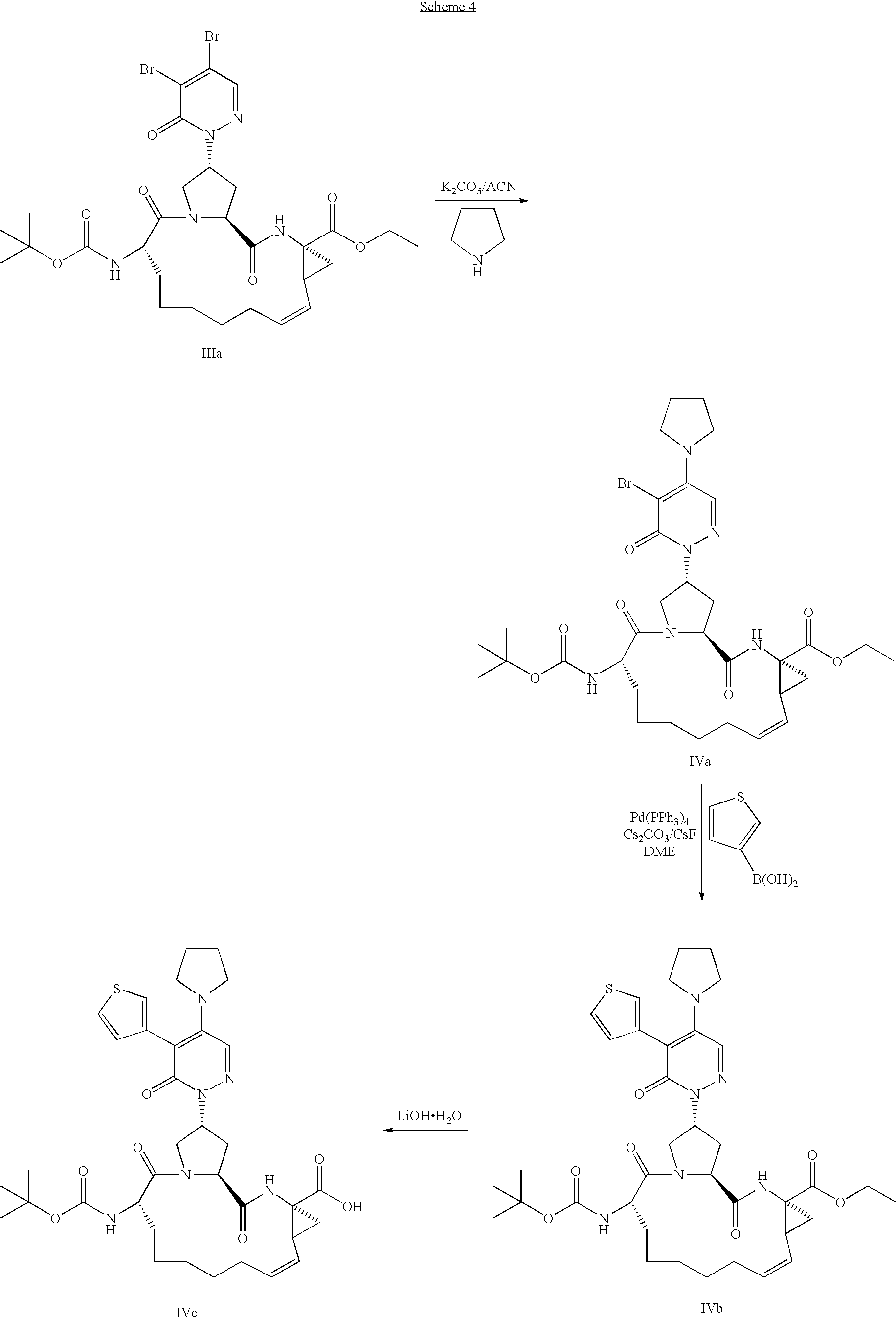

Differentiation between the bromides on macrocyclic IIIa is achieved via Michael addition. As shown in Scheme 4, commercially available pyrrolidine is coupled with di-bromide IIIa to give compound IVa in 87% yield. The bromide moiety a to the carbonyl is then under goes a Suzuki coupling reaction with 3-thiophene boronic acid to produce intermediate IVb, which is further treated with LiOH to afford analog IVc. For further details concerning the Suzuki coupling reaction see A. Suzuki, Pure Appl. Chem. 63, 419-422 (1991) and A. R. Martin, Y. Yang, Acta Chem. Scand. 47,221-230 (1993).

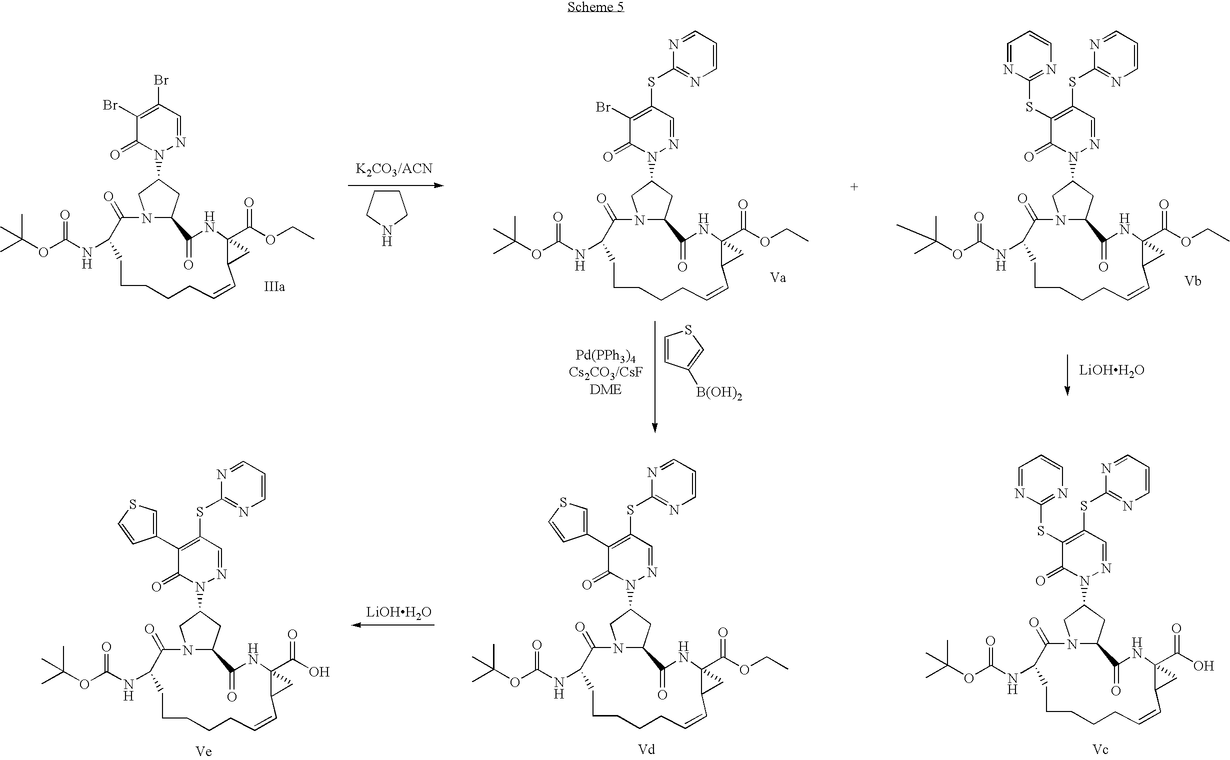

While the secondary amine nucleophile pyrrolidine gave exclusive addition to the 5-bromide position on macrocycle IIIa, sulfur-containing nucleophiles did not exhibit the same selectivity as shown in Scheme 5. With sulfur-containing nucleophiles, addition on both bromines of IIIa is observed together with the mono-coupled product Va with only one equivalent of mercaptopyrimidine. The separability of compounds Va, Vb and starting material IIIa by flash column chromatography allowed for a further Suzuki coupling of the mono-alkylated Va with 3-thiophene boronic acid followed by hydrolysis of Vd with LiOH to furnish analog Ve. The di-alkylated product Vb is also hydrolyzed with LIOH to produce analog Vc.

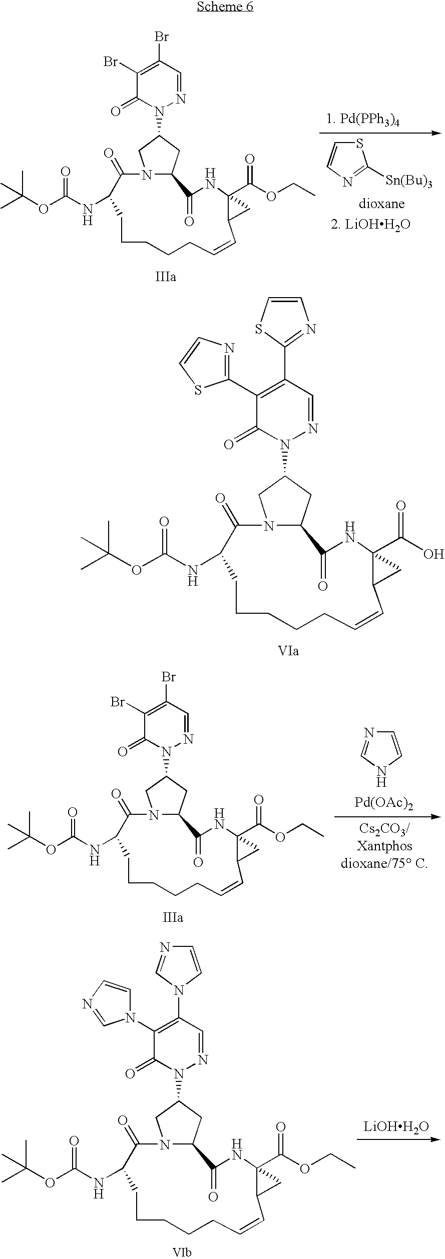

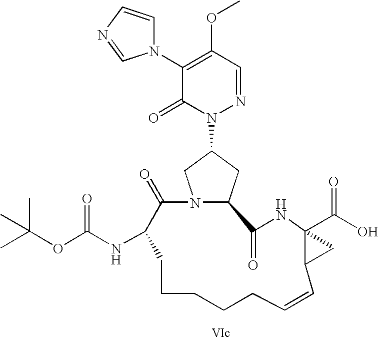

With only a limited number of boronic acids available for Suzuki coupling, other coupling methods such as Stille coupling and N-arylation using Buchwald's chemistry were also explored (Scheme 6). Coupling of intermediate IIIa with 2-stannylthiazole with Stille standard conditions followed by hydrolysis afforded analog VIa. As for N-arylation, coupling of imidazole to di-bromide 6 proceeded smoothly. Unfortunately, hydrolysis with LiOH resulted in replacement of the imidazole moiety on position 5 with a methoxy (VIb. For further details concerning Stille coupling reactions see J. K. Stille, Angew. Chem. Int. Ed. 25, 508-524 (1986); M. Pereyre etal., Tin in Organic Synthesis (Butterworths, Boston, 1987) pp 185-207 passim., and T. N. Mitchell, Synthesis 1992, 803-815. For further details of the Buchwald reaction see J. F. Hartwig, Angew. Chem. Int. Ed. 37, 2046-2067 (1998).

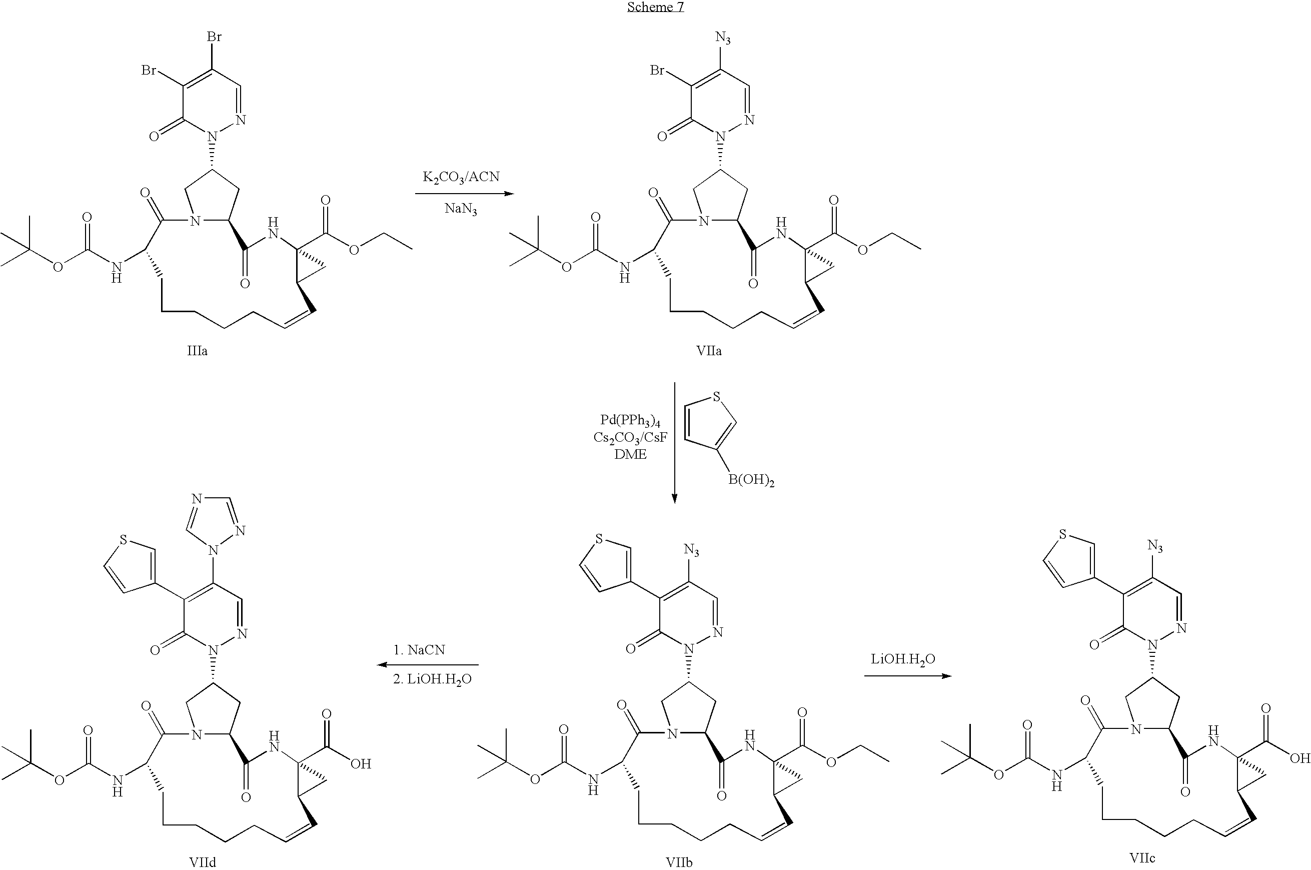

Another method for diversifying pyridazinone analogs is outlined in Scheme 7. Michael addition with sodium azide as the nucleophile to di-bromo IIIa yielded, as in the secondary amine case, only the mono-coupled compound VIIa. Further Suzuki coupling with 3-thiophene boronic acid produced azide VIIb. Compound VIIb is hydrolyzed to give analog VIIc. In addition, the azide moiety of compound VIIb is further converted to tetrazole under standard conditions with sodium cyanide, followed by hydrolysis to provide analog VIId.

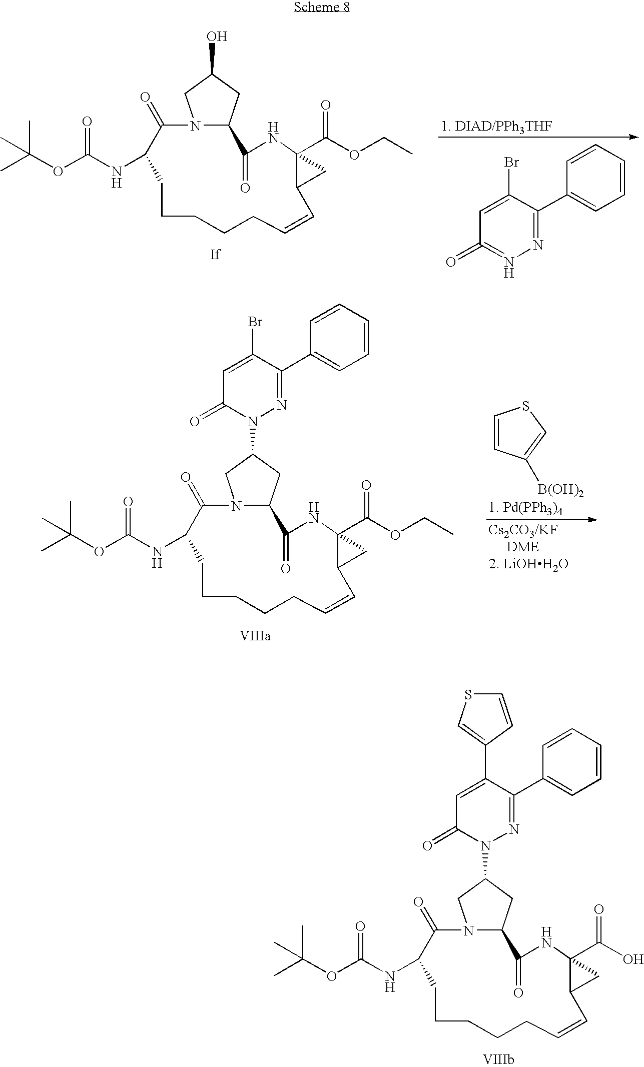

The synthesis of 5,6 pyridazinonyl macrocycle VIIIb is outlined in Scheme 8. Commercially available 5-bromo-6-phenyl-2H-pyridazin-3-one is condensed with key intermediate If via Mitsunobu conditions to give compound VIIIa. Product VIIIa is further subjected to Suzuki coupling conditions with 3-thiophene boronic acid, followed by hydrolysis to give the desired analog VIIIb.

### EXAMPLES

The compounds and processes of the present invention will be better understood in connection with the following examples, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims

### Example 1

Synthesis of the Cyclic Peptide Precursor

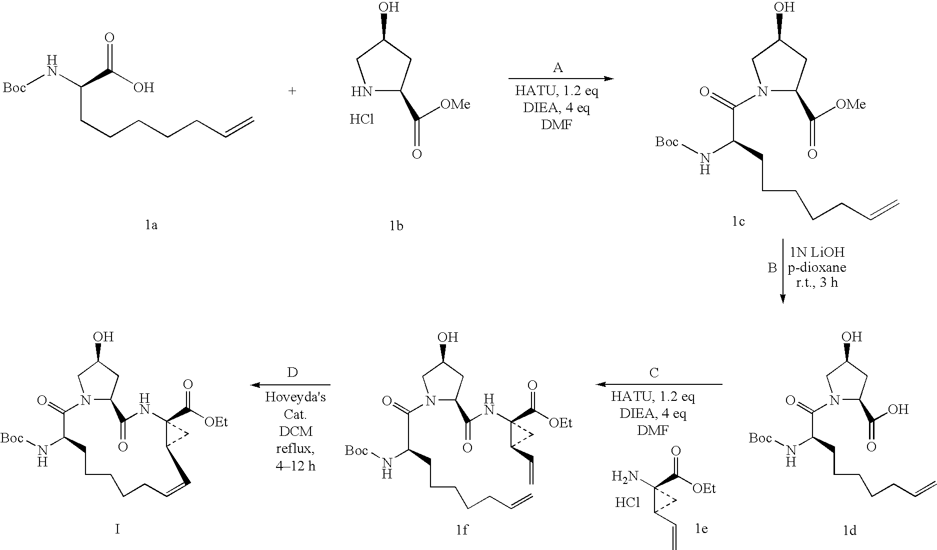

1 A. To a solution of Boc-L-2-amino-8-nonenoic acid 1a (1.36 g, 5 mol) and the commercially available cis-L-hydroxyproline methyl ester 1b (1.09 g, 6 mmol) in 15 ml DMF, DIEA (4 ml, 4 eq.) and HATU (4 g, 2 eq) were added. The coupling is carried out at 0° C. over a period of 1 hour. The reaction mixture is diluted with 100 mL EtOAc, and followed by washing with 5% citric acid 2×20 ml, water 2×20 ml, 1M NaHCO₃ 4×20 ml and brine 2×10 ml, respectively. The organic phase is dried over anhydrous Na₂SO₄ and then is evaporated, affording the dipeptide 1c (1.91 g, 95.8%) that is identified by HPLC (Retention time=8.9 min, 30-70%, 90% B), and MS (found 421.37, M+Na⁺).

1B. The dipeptide 1c (1.91 g) is dissolved in 15 mL of dioxane and 15 mL of 1 N LiOH aqueous solution and the hydrolysis reaction is carried out at RT for 4hour. The reaction mixture is acidified by 5% citric acid and extracted with 100 mL EtOAc, and followed by washing with water 2×20 ml, 1 M NaHCO₃ 2×20 ml and brine 2×20 ml, respectively. The organic phase is dried over anhydrous Na₂SO₄ and then removed in vacuum, yielding the free carboxylic acid compound 1d (1.79 g, 97%), which is used for next step synthesis without need for further purification.

1C. To a solution of the free acid obtained above (1.77, 4.64 mmol) in 5 ml DMF, D-β-vinyl cyclopropane amino acid ethyl ester 1e (0.95 g, 5 mmol), DIEA (4 ml, 4 eq.) and HATU (4 g, 2 eq) were added. The coupling is carried out at 0° C. over a period of 5hour. The reaction mixture is diluted with 80 mL EtOAc, and followed by washing with 5% citric acid 2×20 ml, water 2×20 ml, 1 M NaHCO₃ 4×20 ml and brine 2×10 ml, respectively. The organic phase is dried over anhydrous Na₂SO₄ and then evaporated. The residue is purified by silica gel flash chromatography using different ratios of hexanes:EtOAc as elution phase (5:1→3:1→1:1→1:2→1:5). The linear tripeptide 1f is isolated as an oil after removal of the elution solvents (1.59 g, 65.4%), identified by HPLC (Retention time=11.43 min) and MS (found 544.84, M+Na⁺).

1 D. Ring Closing Metathesis (RCM). A solution of the linear tripeptide 1f (1.51 g, 2.89 mmol) in 200 ml dry DCM is deoxygenated by bubbling N₂. Hoveyda's 1^{st} generation catalyst (5 mol % eq.) is then added as solid. The reaction is refluxed under N₂ atmosphere 12 hours. The solvent is evaporated and the residue is purified by silica gel flash chromatography using different ratios of hexanes:EtOAc as elution phase (9:1 5:1→3:1→1:1 1:2→1:5). The cyclic peptide precursor 1 is isolated as a white powder after removal of the elution solvents (1.24 g, 87%), identified by HPLC (Retention time=7.84 min, 30-70%, 90% B), and MS (found 516.28, M+Na⁺). For further details of the synthetic methods employed to produce the cyclic peptide precursor Ig, see WO 00/059929 (2000), which is herein incorporated by reference in its entirety.

### Example 2

Compound of Formula I, wherein A=tBOC, G=OEt, L=Absent, X═Y=Bromo, Z=Hydrogen, j=3, m=s=1, and R³═R⁴=Hydrogen.

To a mixture of macrocyclic compound 1 (185 mg, 0.38 mmol), 4,5-dibromo-2H-pyridazin-3-one (95 mg, 0.38 mmol) and triphenylphosphine (197 mg, 0.75 mmol) in THF (5 mL) is added DIAD (148 μL, 0.75 mmol) dropwise at 0° C. After stirring at 0° C. for 15 min., the solution is warmed to room temperature and is further stirred for 16 hours. The mixture is then concentrated in vacuo and the residue is purified by column chromatography eluting with 40% ethyl acetate-hexane to give 235 mg (86%) of the title compound.

¹H-NMR (500 MHz, CDCl₃) δ (ppm): 7.8 (s, 1H), 7.1 (brs, 1H), 5.5 (m, 2H), 5.2 (m, 2H), 5.0 (m, 1H), 4.4 (brt, 1H), 4.0-4.2 (m, 4H), 2.9 (m, 1H), 2.6 (m, 1H), 1.8-2.3 (m, 5H), 1.4 (s, 9H), 1.2 (t, 3H). [M+H]⁺=730.6.

### Example 3

Compound of Formula I, wherein A=tBOC, G=OEt, L=Absent, X═Y=Thiophen-3-yl, Z=Hydrogen, j=3, m=s=1, and R³═R⁴=Hydrogen.

A mixture of the title compound of Example 2 (40 mg, 0.055 mmol), 3-thiophene boronic acid (35 mg, 0.28 mmol), cesium carbonate (71 mg, 0.22 mmol), potassium fluoride monohydrate (41 mg, 0.44 mmol) is placed in a round bottom flask and is flushed twice with nitrogen. To this mixture is added DME and the resulting solution is flushed again with nitrogen before palladium tetrakis(triphenylphopshine) (7 mg, 10 mol %) is added. After flushing two more times with nitrogen, the mixture is heated to reflux for 20 hours. The mixture is then cooled and then diluted with water and extracted three times with EtOAc. The combined EtOAc layers are washed once with brine, dried (MgSO₄), filtered and concentrated in vacuo. The residue is purified by column chromatography eluting with 20-40% EtOAc-hexane to give the title compound as a clear film (24 mg, 60%).

¹H-NMR (500 MHz, CDCl₃) δ (ppm): 7.9 (s, 1H), 7.6 (s, 1H), 7.3 (s, 1H), 7.3 (m, 1H), 7.0 (s, 1H), 6.9 (d, 1H), 6.8 (d, 1H), 5.7 (m, 1H), 5.5 (m, 1H), 5.4 (brd, 1H), 5.2 (t, 1H), 5.0 (m, 1H), 4.6 (brt, 1H), 4.0-4.2 (m, 4H), 2.9 (m, 1H), 2.6 (m, 1H), 2.0-2.3 (m, 5H), 1.4 (s, 9H), 1.2 (t, 3H). [M+Na]⁺=758.63.

### Example 4

Compound of Formula I, wherein A=tBOC, G=OH, L=Absent, X═Y=Thiophen-3-yl, Z=Hydrogen, j=3, m=s=1, and R³═R⁴=Hydrogen.

To a solution of the title compound in Example 2 (24 mg, 0.033 mmol) in THF/MeOH/H₂O (2/1/0.5 mL) is added lithium hydroxide (14 mg, 0.33 mmol). After stirring for 16 hours at room temperature, the mixture is acidified to pH 4 with citric acid and extracted three times with EtOAc. The combined organic extracts are washed once with brine, dried (MgSO₄), filtered and concentrated in vacua. The residue is purified by column chromatography eluting with 5-10% methanol-chloroform to give the title compound (13 mg, 56%).

[M+H]⁺=708.3.

### Example 5

Compound of Formula I, wherein A=tBOC, G=OH, L=Absent, X═Y=Phenyl, Z=Hydrogen, j=3, m=s=1, and R³═R⁴=Hydrogen.

The title compound is prepared by a double Suzuki coupling with phenylboronic acid and the title compound of Example 2 according to the procedure set forth in Example 3, followed by hydrolysis of the ethyl ester via the method described in Example 4.

[M+H]⁺=696.40

### Example 6

Compound of Formula I, wherein A=tBOC, G=OH, L=Absent, X═Y=4-(N,N-dimethylamino)phenyl, Z=Hydrogen, j=3, m=s=1, and R³═R⁴=Hydrogen.

The title compound is prepared by a double Suzuki coupling with 4-(N,N-dimethylamino)phenyl boronic acid and the title compound of Example 2 according to the procedure set forth in Example 3, followed by hydrolysis of the ethyl ester via the method described in Example 4.

[M+H]⁺=782.30

### Example 7

Compound of Formula I, wherein A=tBOC, G=OH, L=Absent, X═Y=4-(trifluoromethoxy)phenyl, Z=Hydrogen, j=3, m=s=1, and R³═R⁴=Hydrogen.

The title compound is prepared by a double Suzuki coupling with 4-(trifluoromethoxy)phenyl boronic acid and the title compound of Example 2 according to the procedure set forth in Example 3, followed by hydrolysis of the ethyl ester via the method described in Example 4.

[M+H]⁺=864.09

### Example 8

Compound of Formula I, wherein A=tBOC, G=OH, L=Absent, X═Y=4-(methanesulfonvl)phenyl, Z=Hydrogen, j=3, m=s=1, and R³═R⁴=Hydrogen.

The title compound is prepared by a double Suzuki coupling with 4-(methanesulfonyl)phenyl boronic acid and the title compound of Example 2 according to the procedure set forth in Example 3, followed by hydrolysis of the ethyl ester via the method described in Example 4.

### Example 9

Compound of Formula I, wherein A=tBOC, G=OH, L=Absent, X═Y=4-(cyano)phenyl, Z=Hydrogen, j=3, m=s=1, and R³═R⁴=Hydrogen.

The title compound is prepared by a double Suzuki coupling using 4-cyanophenyl boronic acid and the title compound of Example 2 according to the procedure set forth in Example 3, followed by hydrolysis of the ethyl ester via the method described in Example 4.

[M+H]⁺=746.14

### Example 10

Compound of Formula I, wherein A=tBOC, G=OH, L=Absent, X═Y=pyrid-3-yl, Z=Hydrogen, j=3, m=s=1, and R³═R⁴=Hydrogen.

The title compound is prepared by a double Suzuki coupling using 3-pyridyl boronic acid and the title compound of Example 2 according to the procedure set forth in Example 3, followed by hydrolysis of the ethyl ester via the method described in Example 4.

[M+H]⁺=698.3.

### Example 11

Compound of Formula I, wherein A=tBOC, G=OH, L=Absent, X═Y=4-(morpholin-4-yl-methanonyl)phenyl, Z=Hydrogen, j=3, m=s=1, and R³═R⁴=Hydrogen.

The title compound is prepared by a double Suzuki coupling using 4-carboxyphenyl boronic acid and the title compound of Example 2 according to the procedure set forth in Example 3, followed by amide formation with morpholine, under standard amide bond formation conditions, e.g. PyBrOP, DIEA, and DMAP in DMF. The ethyl ester of the resulting compound is then hydrolyzed via the hydrolysis procedure of Example 4.

### Example 12

Compound of Formula I, wherein A=tBOC, G=OH, L=Absent, X=Bromo, Y=Methoxy, Z=Hydrogen, j=3, m=s=1, and R³═R⁴=Hydrogen.

The title compound is prepared from the title compound in Example 2 via hydrolysis of the ethyl ester according to the procedure described in Example 4, however addition of methoxy to the 5 position is observed in addition to hydrolysis of the ethyl ester.

[M+H]⁺=652.2, 654.2.

### Example 13

Compound of Formula I, wherein A=tBOC, G=OH, L=Absent, X and Y Taken Together=Phenyl, Z=4-methoxyphenyl, j=3, m=s=1, and R³═R⁴=Hydrogen.

The title compound is prepared according to the Mitsunobu conditions set forth in Example 2 with commercially available 4-(4-methoxy-phenyl)-2H-phthalazin-1-one, and subsequent hydrolysis of the ethyl ester via the procedure of Example 4.

[M+H]⁺=700.1.

### Example 14

Compound of Formula I, wherein A=tBOC, G=OH, L=Absent, X and Y Taken Together=Phenyl, Z=4-chlorophenyl, j=3, m=s=1, and R³═R⁴=Hydrogen.

The title compound is prepared according to the Mitsunobu conditions set forth in Example 2 with commercially available 4-(4-chloro-phenyl)-2H-phthalazin-1-one, and subsequent hydrolysis of the ethyl ester via the procedure of Example 4.

[M+H]⁺=704.2.

### Example 15

Compound of Formula I, wherein A=tBOC, G=OH, L=Absent, X=4-fluorophenyl, Y=Hydrogen, Z=Phenyl, j=3, m=s=1, and R³═R⁴=Hydrogen.

The title compound is prepared according to the Mitsunobu conditions set forth in Example 2 with commercially available 4-(4-fluoro-phenyl)-6-phenyl-2H-pyridazin-3-one, and subsequent hydrolysis of the ethyl ester via the procedure of Example 4.

[M+H]⁺=704.2.

### Example 16

Compound of Formula I, wherein A=tBOC, G=OH, L=Absent, X=Hydrogen, Y=1-piperidyl, Z=Phenyl, j=3, m=s=1, and R³═R⁴=Hydrogen.

The title compound is prepared according to the Mitsunobu conditions set forth in Example 2 with commercially available 6-phenyl-5-piperidin-1-yl-2H-pyridazin-3-one, and subsequent hydrolysis of the ethyl ester via the procedure of Example 4.

[M+H]⁺=702.3.

### Example 17

Compound of Formula I, wherein A=tBOC, G=OEt, L=Absent, X=Hydrogen, Y=Bromo, Z=Phenyl, j=3, m=s=1, and R³═R⁴=Hydrogen.

The title compound is prepared according to the Mitsunobu conditions set forth in Example 2 with commercially available 5-Bromo-6-phenyl-2H-pyridazin-3-one.

[M+H]⁺=726.3, 728.3.

### Example 18

Compound of Formula I, wherein A=tBOC, G=OH, L=Absent, X=Hydrogen, Y=Thiophen-3-yl, Z=Phenyl, j=3, m=s=1, and R³═R⁴=Hydrogen.

The title compound is prepared with the title compound of Example 17 and thiophen-3-yl boronic acid according to the Suzuki coupling conditions described in Example 3, followed by the hydrolysis of the ethyl ester via the method described in Example 4.

[M+H]⁺=730.3

### Example 19

Compound of Formula I, wherein A=tBOC, G=OEt, L=Absent, X=Bromo, Y=1-pyrrolidyl, Z=Hydrogen, j=3, m=s=1, and R³═R⁴=Hydrogen.

A mixture of the title compound in Example 2 (45 mg, 0.062 mmol), pyrrolidine (21 mL, 0.25 mmol), and potassium carbonate (34 mg, 0.25 mmol) in 2 mL of acetonitrile is heated to reflux for 3 hours. After cooling to room temperature, the mixture is filtered through a sinter glass funnel and the filtrate is concentrated in vacuo. The residue is re-dissolved in ethyl acetate and then washed once with saturated sodium carbonate, once with brine, dried (MgSO₄), filtered, and concentrated under vacuum to give a yellow residue which is chromatographed over silica gel eluting with 3% methanol-chloroform to give 37 mg (83%) of the title compound.

[M+H]⁺=719.2, 721.2.

### Example 20

Compound of Formula I, wherein A=tBOC, G=OH, L=Absent, X=Thiophen-3-yl, Y=1-pyrrolidyl, Z=Hydrogen, j=3, m=s=1, and R³═R⁴=Hydrogen.

The title compound is prepared with the title compound in Example 19 and thiephen-3-yl boronic acid using the Suzuki conditions described in Example 3, followed by hydrolysis of the ethyl ester according to the method set forth in Example 4.

[M+H]⁺=694.3.

### Example 21

Compound of Formula I, wherein A=tBOC, G=OEt, L=Absent, X=Bromo, Y=Azido, Z=Hydrogen, j=3, m=s=1, and R³═R⁴=Hydrogen.

A mixture of the title compound in Example 2 (45 mg, 0.062 mmol), sodium azide (16 mg, 0.25 mmol), and potassium carbonate (34 mg, 0.25 mmol) in 2 mL of acetonitrile is heated to reflux for 3 hours. After cooling to room temperature, the mixture is filtered through a sinter glass funnel and the filtrate is concentrated in vacuo. The residue is re-dissolved in ethyl acetate and then washed once with saturated sodium carbonate, once with brine, dried (MgSO₄), filtered, and concentrated under vacuum to give a yellow residue which is chromatographed over silica gel eluting with 3% methanol-chloroform to give 37 mg (83%) of the title compound.

### Example 22

Compound of Formula I, wherein A=tBOC, G=OEt, L=Absent, X=Thiophen-3-yl, Y=Azido, Z=Hydrogen, j=3, m=s=1, and R³═R⁴=Hydrogen.

The title compound is prepared with the title compound in Example 21 and thiophen-3-yl boronic acid using the Suzuki conditions described in Example 3.

### Example 23

Compound of Formula I, wherein A=tBOC, G=OH, L=Absent, X=Thiophen-3-yl, Y=Azido, Z=Hydrogen, j=3, m=s=1, and R³═R⁴=Hydrogen.

The title compound is prepared by hydrolysis of the ethyl ester of the title compound of Example 22 via the hydrolysis procedure of Example 4.

### Example 24

Compound of Formula I, wherein A=tBOC, G=OH, L=Absent, X=Thiophen-3-yl, Y=Tetrazol-2-yl, Z=Hydrogen, j=3, m=s=1, and R³═R⁴=Hydrogen.

To a solution of the title compound of Example 22 (2.63 mmol) in toluene (8 ml) is added KCN (10.53 mmol) and Et₃N.HCl (10.53 mmol). The mixture is heated at 115° C. for 18 hrs, diluted with DCM, washed with 5% citric acid (aq), dried over anhydrous Na₂SO₄, and concentrated in vacuo to afford the ethyl ester of the title compound in crude form. Hydrolysis of the ethyl ester via the method described in Example 4 yields the title compound.

### Example 25

Compound of Formula I, wherein A=tBOC, G=OH, L=Absent, X═Y=Mercapto-2-pryrimidine, Z=Hydrogen, j=3, m=s=1, and R³═R⁴=Hydrogen.

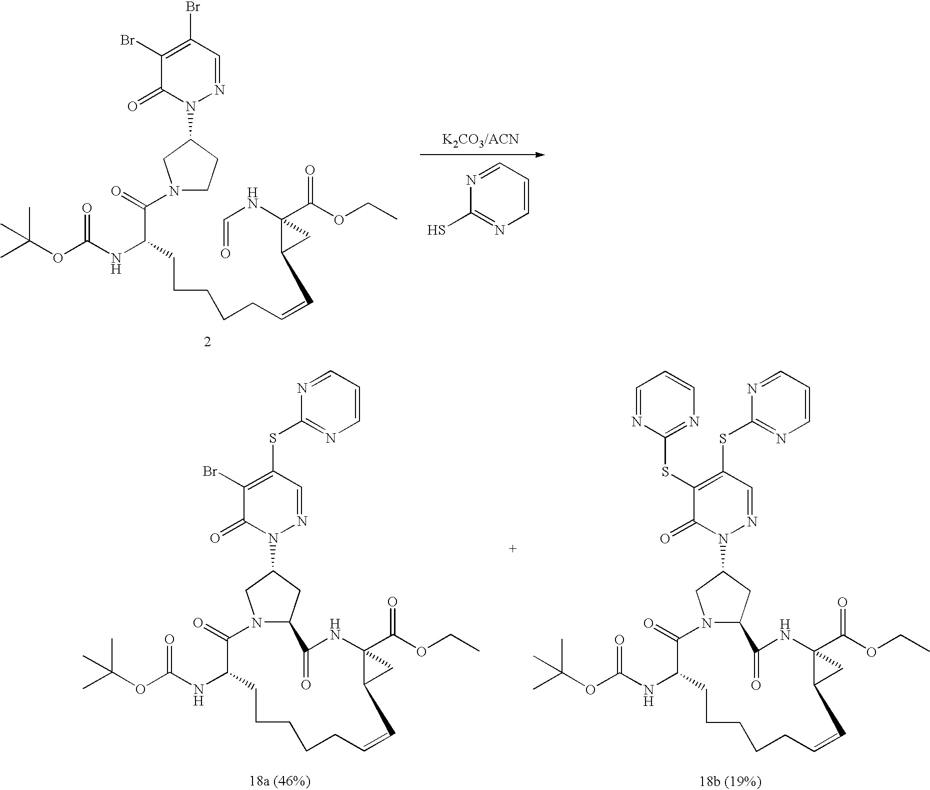

A mixture of the title compound in Example 2 (45 mg, 0.062 mmol), pyrimidine-2-thiol (0.25 mmol), and potassium carbonate (34 mg, 0.25 mmol) in 2 mL of acetonitrile is heated to reflux for 3 hours. After cooling to room temperature, the mixture is filtered through a sinter glass funnel and the filtrate is concentrated in vacuo. The residue is re-dissolved in ethyl acetate and then washed once with saturated sodium carbonate, once with brine, dried (MgSO₄), filtered, and concentrated under vacuum to give a yellow residue which is chromatographed over silica gel eluting with 3% methanol-chloroform to afford 18b in a 19% yield.

The ethyl ester of compound 18b is then hydrolyzed via the method described in Example 4 to give the title compound.

[M+H]⁺=764.3.

### Example 26

Compound of Formula I, wherein A=tBOC, G=OH, L=Absent, X=Bromo, Y=Mercapto-2-pryrimidine, Z=Hydrogen, j=3, m=s=1, and R³═R⁴=Hydrogen.

The title compound is prepared by hydrolysis of the ethyl ester of compound 18a, formed in Example 25, via the method set forth in Example 4.

[M+H]⁺=732.2, 734.2.

### Example 27

Compound of Formula I, wherein A=tBOC, G=OH, L=Absent, X=Thiophen-3-yl, Y=Mercapto-2-pryrimidine, Z=Hydrogen, j=3, m=s=1, and R³═R⁴=Hydrogen.

The title compound is prepared with compound 18a from Example 25 and thiophen-3-yl boronic acid according to the Suzuki coupling conditions set forth in Example 3, followed by hydrolysis of the ethyl ester via the method described in Example 4.

### Example 28

Compound of Formula I, wherein A=tBOC, G=OEt, L=Absent, X═Y=thiazol-2-yl, Z=Hydrogen, j=3, m=s=1, and R³═R⁴=Hydrogen.

To a degassed solution of the title compound of Example 2 (1 mmol) and thiazol-2-yl stannane (2 mmol) is added Pd(PPh₃)₄ (10 mol %). The mixture is degassed with nitrogen 2 more times and is heated to 100° C. for 3 hour. The cooled mixture is concentrated under vacuum and the residue is purified by column chromatography eluting with 30% EtOAc/Hexane followed by the hydrolysis of the ethyl ester via the method of Example 4 to give the title compound.

[M+H]⁺=710.3.

### Example 29

Compound of Formula I, wherein A=tBOC, G=OH, L=Absent, X═Y=Imidazol-1-yl, Z=Hydrogen, j=3, m=s=1, and R³═R⁴=Hydrogen.

The title compound is prepared by adding to a dry mixture of the title compound from Example 2 (0.068 mmol), imidazole (2 eq.), Cs₂CO₃ (3 eq.), Xantphos (30 mol %), and Pd(OAc)₂ under nitrogen dioxane. The reaction mixture is then degassed and stirred at 75° C. for 18 hours. Upon completion of the reaction, monitored via TLC, the reaction mixture is diluted with DCM, filtered, and concentrated in vacuo. The reaction mixture is then purified via silica column chromatography with 5% MeOH/CHCl₃ to afford the ethyl ester of the title compound. The ethyl ester is then hydrolyzed by the conditions set forth in Example 4 to afford the title compound.

### Example 30

Compound of Formula I, wherein A=tBOC, G=OH, L=Absent, X=2-(cyclopropylamino)-thiazol-4-yl, Y=4-methoxyphenyl, Z=Hydrogen, j=3, m=s=1, and R³═R⁴=Hydrogen.

Formation of 4-(2-Cyclopropylamino-thiazol-4-yl)-5-(4-methoxy-phenyl)-2H-pyridazin-3-one (30h)

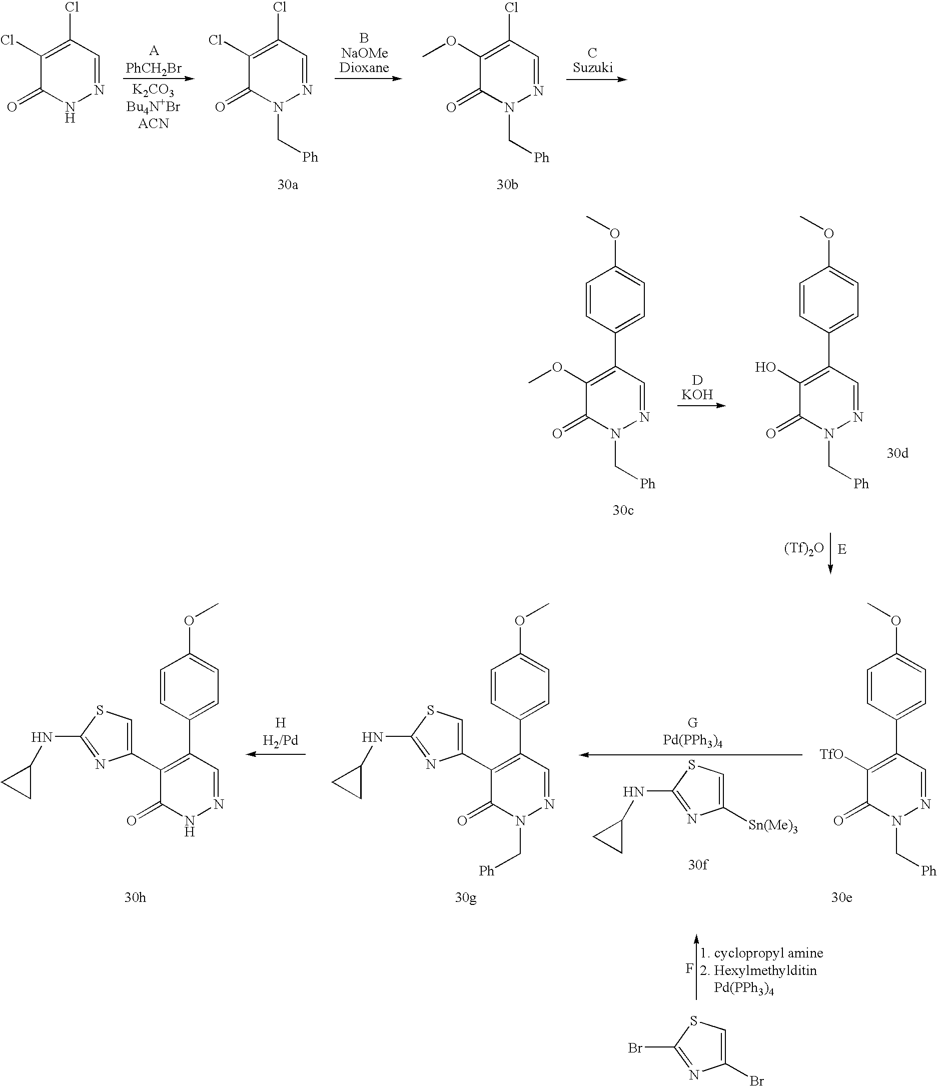

30A. A mixture of commercially available 4,5-dichloropyridazin3(2H)-one (18 mmol), benzyl bromide (19 mmol), potassium carbonate (45 mmol), tetrabutylammonium bromide (1 mmol) and acetonitrile (45 mL) is stirred and heated under reflux for 1 h. After cooling, the solvent is evaporated under reduced pressure. The residue is purified by filtration on a small silica gel column eluting with 10% EtOAc/Hexane to give compound 30a as a white powder (81%). [M+H]⁺=256.3.

30B. To a magnetically stirred solution of 30a (4.5 mmol) in dry dioxane (20 mL) is added 1.0 mL of 21 wt % solution of sodium methoxide at room temperature. After 1 hour, the mixture is poured into water/ethyl acetate and the organic layer is dried over MgSO₄ and concentrated to an oil. The oil residue is purified by column chromatography eluting with 10% EtOAc/Hex to give 85% of 30b.

[M+H]⁺=251.7.

Alternate substitution of pyridazinone 30b can be achieved via this step using MeOH rather than dioxane as a solvent, wherein the methoxy occupies the 5 position on the pyridazinone ring and the chloro resides at the 4 position.

30C. Pyridazinone 30b (1 mmol) is dissolved in DME. To this mixture is added Pd(PPh₃)₄ (10 mol %) and the mixture is stirred at room temperature for 10 min before 4 methoxybenzeneboronic acid (2 mmol) and aqueous 1 mL of Na₂CO₃ (10 wt %) are added. Subsequently, the reaction mixture is heated to reflux for 18 hours. The cooled reaction mixture is diluted with water and extracted 3 times with ethyl acetate. The combined organic layers are dried (MgSO₄), filtered and concentrated under vacuum. The residue is purified by column chromatography on silica gel eluting with 15% EtOAc/Hexane to give compound 30c. [M+H]⁺=323.3.

30D. To a solution of 30c (3 mmol) in DME is added 2N KOH and the resulting mixture is heated to reflux for 1 hour. The cooled mixture is diluted with water and acidified with solid citric acid to pH ˜5 and extracted 3 times with CH₂Cl₂. The organic layers are washed once with brine, dried (MgSO₄), filtered and concentrated under vacuum to give compound 30d. [M+H]⁺=309.3.

30E. To a cooled solution of compound 30d (2 mmol), triethylamine (0.4 mL) in dichloromethane (10 mL) (ice-acetone bath) is added trifluoromethanesulfonic anhydride (0.4 mL) dropwise. The resulting solution is stirred for 30 min at −5° C. The reaction mixture is then poured into dilute HCl (0.5 M) and extracted with CH₂Cl₂. The combined organic layers are washed with a 1% NaHCO₃, brine and dried (MgSO₄), filtered and concentrated under vacuum to give a brown oil. Compound 30e is used immediately without further purification. [M+H]⁺=441.4.

30F. Commercially available 2,4-dibromothiazole (2 mmol) is dissolved in cyclopropylamine (3 mL) and the reaction mixture is heated to 50° C. for 8 hour. The cooled mixture is then poured into water and extracted 2 times with ether. After drying the combined organic fractions (MgSO₄), evaporation of solvents, and purification by flash column chromatography (silica gel, 15% EtOAc/Hexane) furnished 2-cyclopropylamine-4-bromothiazole which is further converted to the corresponding stannane 30f. A solution of 2-cyclopropylamine-4-bromothiazole in degassed DME is treated with hexamethylditin and Pd(PPh₃)₄ and heated at 80° C. for 18 hour. The cooled mixture is concentrated under vacuum and the residue is purified by column chromatography eluting with 20% EtOAc/Hexane/2% Et₃N to give Stannane 30f. [M+H]⁺=304.1.

30G. To a degassed solution of compound 30e (1 mmol) and stannane 30f (2 mmol) is added Pd(PPh₃)₄ (10 mol %). The mixture is degassed two additional times with nitrogen and subsequently heated to 100° C. for 3 hour. The cooled mixture is concentrated under vacuum and the residue is purified by column chromatography eluting with 30% EtOAc/Hexane to give compound 30g. [M+H]⁺=431.6.

30H. A solution of compound 30 g and 10% Pd/C (wet) in MeOH is subjected to a hydrogen balloon for 2 hours. The mixture is filtered through a pad of celite and the filtrate is concentrated under vacuum to give compound 30h. [M+H]⁺=341.4.

The title compound is prepared from pyridazinone 30h and the cyclic peptide precursor 1 of Example 1 via the Mitsunobu conditions set forth in Example 3, followed by the hydrolysis of the ethyl ester via the hydrolysis conditions described in Example 4.

### Example 31

Compound of Formula I, wherein A=tBOC, G=OH, L=Absent, X and Y Taken Together=6-methoxy-isoguinolin-(3,4)-yl, Z=Hydrogen, j=3, m=s=1, and R³═R⁴=Hydrogen.

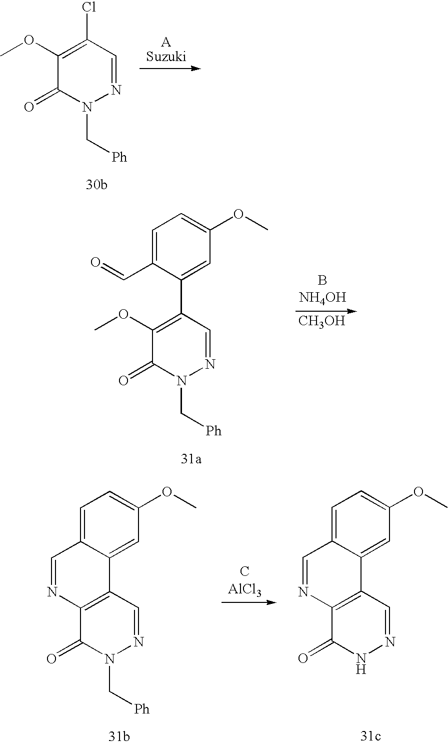

31A. Pyridazinone 30b (2 mmol) is dissolved in DME. To this mixture is added Pd(PPh₃)₄ and the mixture is stirred at room temperature for 10 min before 2-formyl-4-methoxybenzeneboronic acid and aqueous Na₂CO₃ (10 wt %) are added. Subsequently, the reaction mixture is heated to reflux for 18 hours. The cooled reaction mixture is diluted with water and extracted 3 times with ethyl acetate. The combined organic layers are dried (MgSO₄), filtered and concentrated under vacuum. The residue is purified by column chromatography on silica gel eluting with 20% EtOAc/Hexane to give compound 31a. [M+H]⁺=351.4.

31B. A mixture of pyridazinone 31a (1 mmol), MeOH (20 mL) and NH₄OH (10 mL, 28-30 wt %) is heated at 60° C. for 30 min. After cooling, the precipitate, compound 31 b, is filtered and rinsed with MeOH (15 mL). [M+H]⁺=317.4.

31 C. A mixture of pyridazinoisoquinolinone 31b (0.5 mmol), AlCl₃ and toluene is stirred and heated at 70° C. for 1 hour. After cooling, water is added and the mixture is filtered and rinsed with water. The residue is purified by column chromatography on silica gel eluting with 50% EtOAc/Hex to give compound 31 c.

[M+H]⁺=227.3.

The title compound is prepared from pyridazinoisoquinolinone 31c and the cyclic peptide precursor 1 of Example 1 via the Mitsunobu conditions set forth in Example 3, followed by the hydrolysis of the ethyl ester via the hydrolysis conditions described in Example 4.

### Example 32

Compound of Formula I, wherein A=—(C═O)—O—R¹, wherein R¹=Cyclopentyl, G=OH, L=Absent, X═Y=Thiophen-3-yl, Z=Hydrogen, j=3, m=s=1, and R³═R⁴=Hydrogen.

32a—Amine Deprotection.

0.041 mmol of the title compound of Example 3 is dissolved in 4 ml of a 4M solution of HCl in dioxane and stirred for 1 hour. The reaction residue 32a is concentrated in vacua.

32b—Chloroformate Reagent

The chloroformate reagent 32b is prepared by dissolving 0.045 mmol of cyclopentanol in THF (3 ml) and adding 0.09 mmol of phosgene in toluene (20%). The resulting reaction mixture is stirred at room temperature for 2 hours and the solvent is removed in vacua. To the residue is added DCM and subsequently concentrated to dryness twice in vacuo yielding chloroformate reagent 32b.

32c—Carbamate Formation

The title carbamate is prepared by dissolving residue 32a in 1 ml of THF, adding 0.045 mmol of TEA, and cooling the resulting reaction mixture to 0° C. To this 0° C. reaction mixture is added chloroformate reagent 32b in 3 ml of THF. The resulting reaction mixture is reacted for 2 hours at 0° C., extracted with EtOAc, washed by 1 M sodium bicarbonate, water and brine, dried over MgSO₄, and concentrated in vacuo to dryness. The crude compound is purified by silica column and the ethyl ester is subsequently hydrolyzed by the procedure set forth in Example 4.

### Example 33

Compound of Formula I, wherein A=—(C═O)—O—R¹, wherein R¹=Cyclobutyl, G=OH, L=Absent, X═Y=Thiophen-3-yl, Z=Hydrogen, j=3, m=s=1, and R³═R⁴=Hydrogen.

The title compound is prepared by the method described in Example 32 with the title compound of Example 3 and cyclobutanol.

### Example 34

Compound of Formula I, wherein A=—(C═O)—O—R¹, wherein R¹=Cyclohexyl, G=OH, L=Absent, X═Y=Thiophen-3-yl, Z=Hydrogen, j=3, m=s=1, and R³═R⁴=Hydrogen.

The title compound is prepared by the method described in Example 32 with the title compound of Example 3 and cyclohexanol.

### Example 35

Compound of Formula I, wherein A=—(C═O)—O—R¹, wherein R¹=

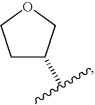

G=OH, L=Absent, X═Y=Thiophen-3-yl, Z=Hydrogen, j=3, m=s=1, and R³═R⁴=Hydrogen.

The title compound is prepared by the method described in Example 32 with the title compound of Example 3 and (R)-3-hydroxytetrahydrofuran.

### Example 36

Compound of Formula I, wherein A=—(C═O)—O—R¹, wherein R¹=

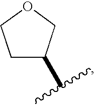

G=OH, L=Absent, X═Y=Thiophen-3-vl, Z=Hydrogen, j=3, m=s=1, and R³═R⁴=Hydrogen.

The title compound is prepared by the method described in Example 32 with the title compound of Example 3 and (S)-3-hydroxytetrahydrofuran.

### Example 37

Compound of Formula I, wherein A=—(C═O)—O—R¹, wherein R¹=

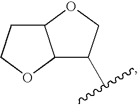

G=OH, L=Absent, X═Y=Thiophen-3-yl, Z=Hydrogen, j=3, m=s=1, and R³═R⁴=Hydrogen.

The title compound is prepared by the method described in Example 32 with the title compound of Example 3 and

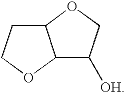

### Example 38

Compound of Formula I, wherein A=—(C═O)—R¹, wherein R¹=Cyclopentyl, G=OH, L=Absent, X═Y=Thiophen-3-yl, Z=Hydrogen, j=3, m=s=1, and R³═R⁴=Hydrogen.

The title compound is prepared with the title compound from Example 3 in 4 ml of a 4M solution of HCl in dioxane and stirring the reaction mixture for 1 hour. The reaction residue is concentrated in vacuo. To this residue, 4 ml of THF and 0.045 mmol of TEA is added, the mixture is cooled to 0° C., to which is added 0.045 mmol of the cyclopentyl acid chloride. The resulting reaction mixture is stirred for 2 hours at 0° C. The reaction mixture is then extracted with EtOAc, washed with 1 M sodium bicarbonate, water and brine, dried over MgSO₄ and concentrated to dryness in vacuo. The crude compound is purified by silica column and the ethyl ester is subsequently hydrolyzed by the procedure set forth in Example 4.

### Example 39

Compound of Formula I, wherein A=—(C═O)—NH—R¹, wherein R¹=cyclopentyl, G=OH, L=Absent, X═Y=Thiophen-3-yl, Z=Hydrogen, j=3, m=s=1, and R³═R⁴=Hydrogen.

The title compound is prepared with the title compound from Example 3 in 4ml of a 4M solution of HCl in dioxane and stirring for 1 hour. The resulting reaction residue is concentrated in vacuo, dissolved in 4 ml THF, and cooled to 0° C. To the 0° C. solution is added 0.045 mmol of cyclopentyl isocyanate and the resulting reaction mixture is stirred at RT for 4 hours. The solution is then extracted with EtOAc, washed with 1% HCl, water and brine, dried over MgSO₄, and concentrated in vacuo to dryness. The crude compound is purified by silica column and the ethyl ester is subsequently hydrolyzed by the procedure set forth in Example 4.

### Example 40

Compound of Formula I, wherein A=—(C═S)—NH—R¹, wherein R¹=Cyclopentyl, G=OH, L=Absent, X═Y=Thiophen-3-yl, Z=Hydrogen, j=3, m=s=1, and R³═R⁴=Hydrogen.

The title compound is prepared with the title compound from Example 3 in 4 ml of a 4M solution of HCl in dioxane and stirring for 1 hour. The resulting reaction residue is concentrated in vacuo, dissolved in 4ml THF, and cooled to 0° C. To the 0° C. solution is added 0.045 mmol of cyclopentyl isothiocyanate and the resulting reaction mixture is stirred at RT for 4 hours. The solution is then extracted with EtOAc, washed with 1% HCl, water and brine, dried over MgSO₄, and concentrated in vacuo to dryness. The crude compound is purified by silica column and the ethyl ester is subsequently hydrolyzed by the procedure set forth in Example 4.

### Example 41

Compound of Formula I, wherein A=—S(O)₂—R¹, wherein R¹=Cyclopentyl, G=OH, L=Absent, X═Y=Thiophen-3-yl, Z=Hydrogen, j=3, m=s=1, and R³═R⁴=Hydrogen.

The title compound is prepared with the title compound from Example 3 in 4 ml of a 4M solution of HCl in dioxane and stirring for 1 hour. To the resulting concentrated reaction residue, which has been dissolved in 4 ml THF, is added 0.045 mmol of TEA, and cooled to 0° C. To the 0° C. solution is added 0.045 mmol of cyclopentyl sulfonyl chloride and the resulting reaction mixture is stirred at 0° C. for 2 hours. The solution is then extracted with EtOAc, washed with 1 M sodium bicarbonate, water and brine, dried over MgSO₄, and concentrated in vacuo to dryness. The crude compound is purified by silica column and the ethyl ester is subsequently hydrolyzed by the procedure set forth in Example 4.

### Example 42

Compound of Formula I, wherein A=—(C═O)—O—R¹, R¹=Cyclopentyl, G=—O-phenethyl, L=Absent, X═Y=Thiophen-3-yl, Z=Hydrogen, j=3, m=s=1, and R³═R⁴=Hydrogen.

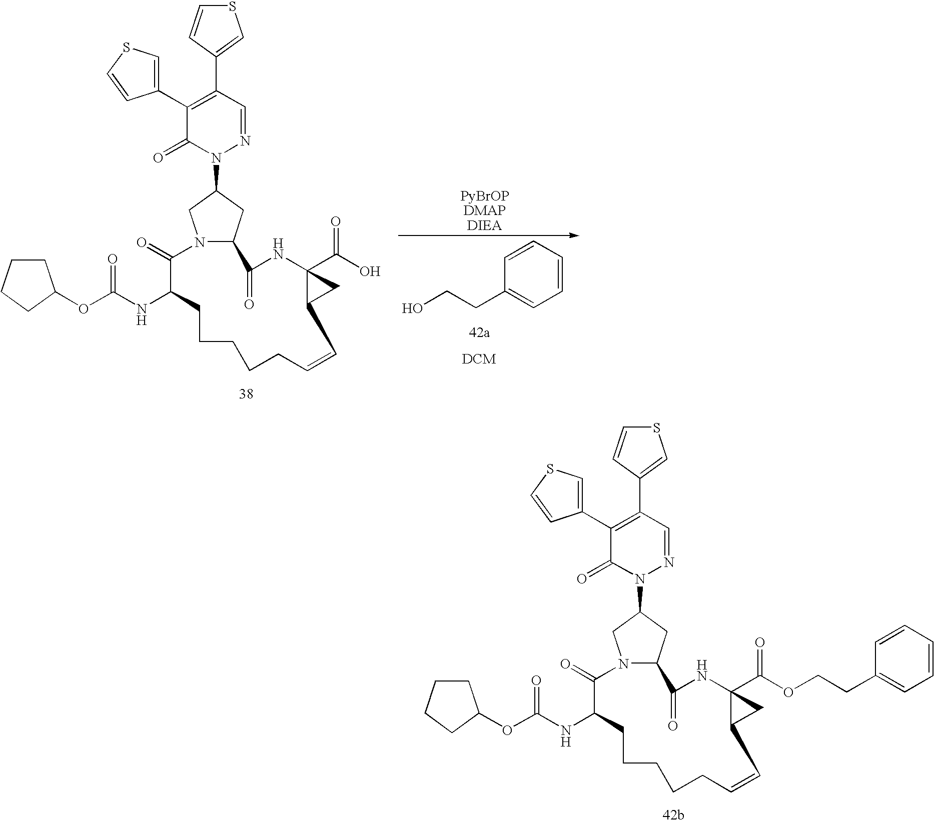

The title compound is prepared by adding to a solution of the title compound of Example 38 and phenethyl alcohol 42a in 0.5 ml DCM, is added 1.2 eq. PyBrOP, 4 eq. DIEA, and catalytic amount of DMAP at 0° C. The resulting reaction mixture is stirred for 1 hour at 0° C. and then warmed to RT over a period of 4-12 hours. The reaction mixture is purified by silica gel flash chromatography using different ratios of hexanes:EtOAc as elution phase (9:1→5:1→3:1→1:1) to afford the title compound isolated phenethyl ester 42b.

Other esters can be made using the same procedures.

### Example 43

Compound of Formula I, wherein A=—(C═O)—O—R¹, R¹=Cyclopentyl, G=—NH-phenethyl, L=Absent, X═Y=Thiophen-3-yl, Z=Hydrogen, j=3, m=s=1, and R³═R⁴=Hydrogen.

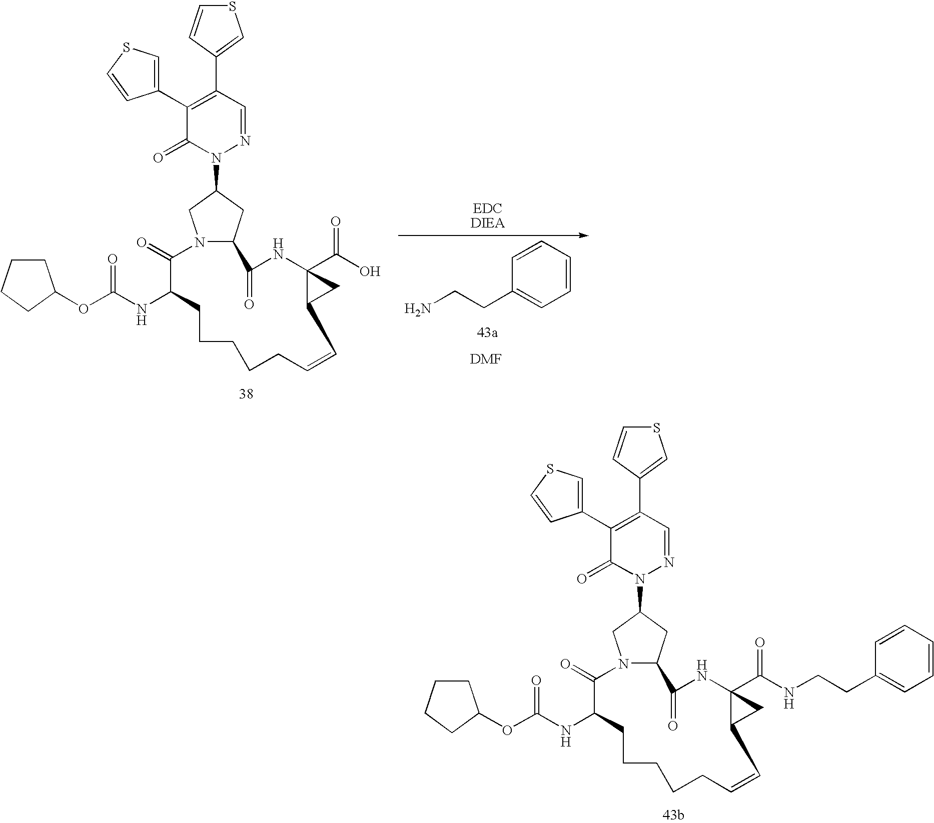

The title compound is prepared by adding to a solution of the title compound of Example 38 and phenethylamine 43a (0.05 ml) in 0.5 ml DMF, EDC (1.2 eq.) and DIEA (4 eq.) at 0° C. The resulting reaction mixture is stirred at 1 hour. Subsequently, the reaction is warmed to RT over a period of 4-12 hours. The reaction mixture is purified by silica gel flash chromatography using different ratios of hexanes:EtOAc as elution phase (9:1→5:1→3:1→1:1) to afford title compound phenethyl amide 43b.

Other amides can be made via the same procedure.

### Example 44

Compound of Formula I, wherein A=—(C═O)—O—R¹, R¹=Cyclopentyl, G=—NHS(O)₂-phenethyl, L=Absent X═Y=Thiophen-3-yl, Z=Hydrogen, j=3, m=s=1, and R³═R⁴=Hydrogen.

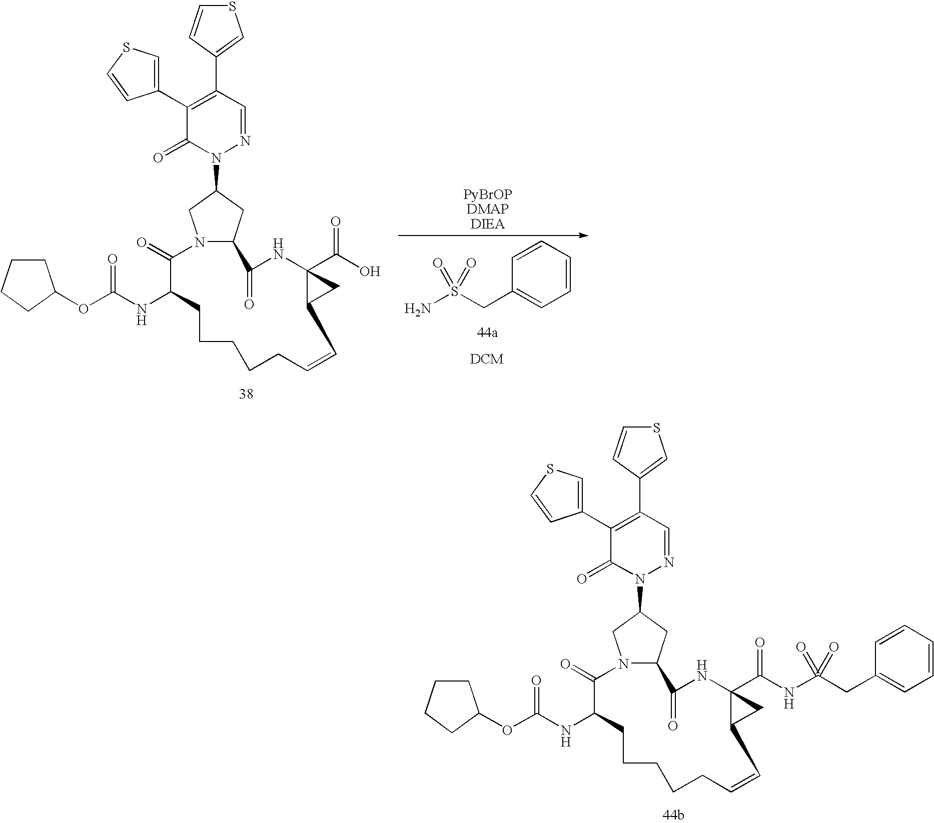

The title compound is prepared by adding to a solution of the title compound of Example 38 and α-toluenesulfonamide 44a (10 mg) in 0.5 ml DCM, is added 1.2 eq. PyBrOP, 4 eq. DIEA, and catalytic amount of DMAP at 0° C. The resulting reaction mixture is stirred for 1 hour and then allowed to warm to RT over a period of 4-12 hours. The reaction mixture is purified by silica gel flash chromatography using different ratios of hexanes:EtOAc as elution phase (9:1→5:1→3:1→1:1) to afford the title compound sulfonamide 44b.

Other sulfonamides can be made via the same procedure.

### Example 45

Compound of Formula I, wherein A=—(C═O)—O—R¹, R¹=Cyclopentyl, G=—(C═O)—OH, L=Absent, X═Y=Thiophen-3-yl, Z=Hydrogen, j=3, m=s=1, and and R³═R⁴=Hydrogen.

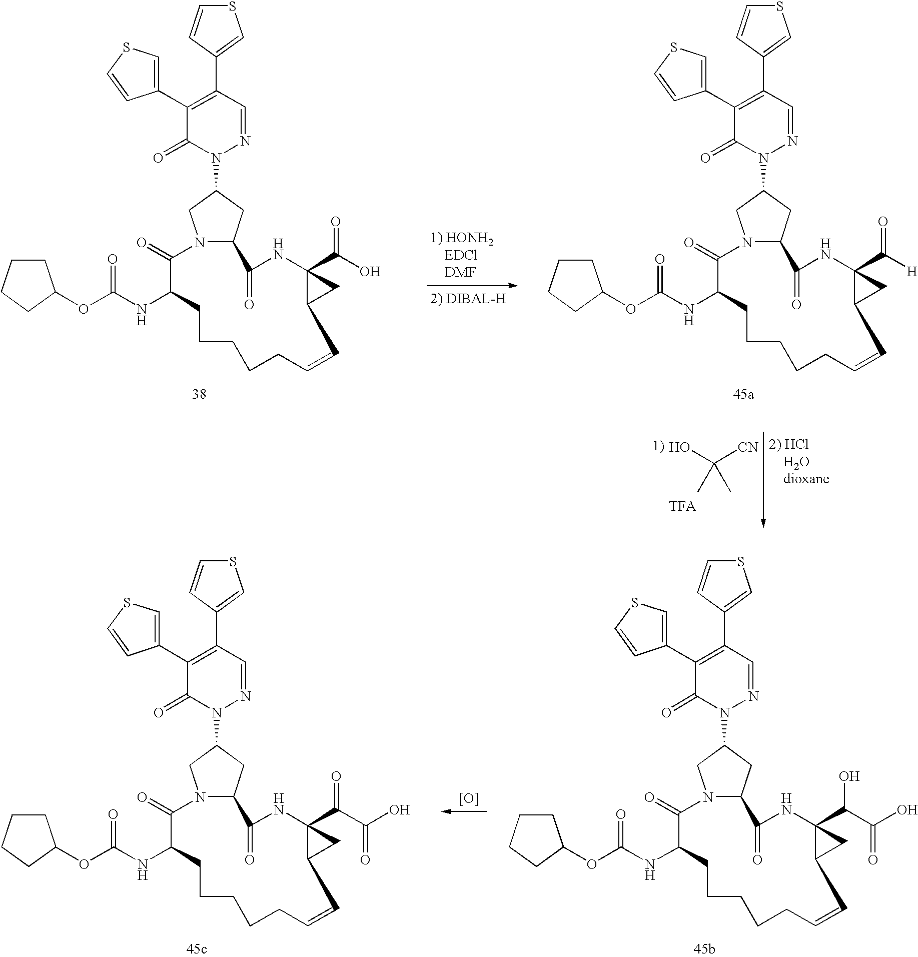

The title compound is prepared by adding to a solution of the title compound of Example 38 in 0.5 ml DMF, EDC (1.2 eq.) and DIEA (4 eq.) at 0° C. The resulting reaction mixture is stirred at 1 hour. Subsequently, the reaction is warmed to RT over a period of 4-12 hours. The reaction mixture is purified by silica gel flash chromatography to afford hydroxyamide. The hydroyamide is then treated with DIBAL-H at -78° C. in THF for 2 hours. The reaction mixture is then diluted with 8 ml EtOAc, washed with water and brine, dried over Na₂SO₄, and concentrated in vacuo to yield aldehyde 45a. To a solution of aldehyde 39a in 0.5 ml THF, is added α-hydroxy-α-methyl-propionitrile (0.1 ml) and catalytic amount TFA at 0° C. The resulting reaction mixture is warmed from 0° C. to RT over a period of 4-12 hours followed by hydrolysis with concentrated hydrochloric acid in dioxane. The reaction is then extracted with EtOAc, and washed with water and brine to yield α-hydroxy compound 45b in its crude form. The crude compound 45b undergoes a Dess-Martin oxidation in THF (0.5 ml), providing the α-carbonyl compound 39c in crude form. The crude 45c is purified by silica gel flash chromatography using different ratios of hexanes:EtOAc as elution phase (9:1→5:1→3:1→1:1) to afford the title compound isolated keto acid 45c.

### Example 46

Compound of Formula I, wherein A=—(C═O)—O—R¹, R¹=Cyclopentyl, G=—(C═O)—O-phenethyl, L=Absent, X═Y=Thiophen-3-yl, Z=hydrogen, j=3, m=s=1, and and R³═R⁴=Hydrogen.

The title compound is prepared with the title compound keto acid of Example 45 and phenethanol according to the procedure set forth in Example 42.

### Example 47

Compound of Formula I, wherein A=—(C═O)—O—R¹, R¹=Cyclopentyl, G=—(C═O)—NH-phenethyl, L=Absent, X═Y=Thiophen-3-yl, Z=hydroqen, j=3, m=s=1, and and R³═R⁴=Hydrogen.

The title compound is prepared with the title compound keto acid of Example 45 and phenethyl amine according to the procedure set forth in Example 43.

### Example 48

Compound of Formula I, wherein A=—(C═O)—O—R¹, R¹=Cyclopentyl, G=—(C═O)—NH—S(O)₂-benzyl, L=Absent, X═Y=Thiophen-3-yl, Z=Hydrogen, j=3, m=s=1, and R³═R⁴=Hydrogen.

The title compound is prepared with the title compound keto acid of Example 45 and α-toluenesulfonamide according to the procedure set forth in Example 44.

### Example 49

Compound of Formula I, wherein A=tBOC, G=OH, L=—(C═O)CH₂, X=Y=Thiophen-3-yl, Z=Hydrogen,=1, m=s=1, and R³═R⁴=Hydrogen.

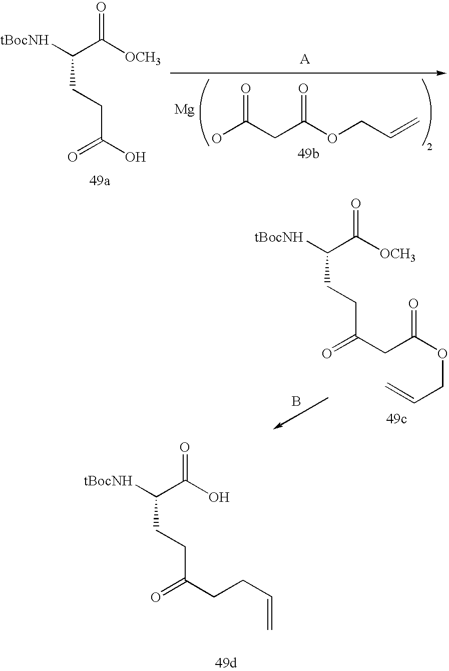

Synthesis of (2S)-N-Boc-amino-5-oxo-non-8-enoic Acid

49A. The aforementioned amino acid is prepared by adding to a solution of monoallyl ester of malonic acid in dry THF under N₂ at −78° C., n-Bu₂Mg dropwise over a period of 5min. The resulting suspension is then stirred at RT for 1 hour and evaporated to dryness. Solid Mg salt 49b, is dried under vacuum.

Glutamic acid derivative 49a is first mixed with 1,1′-carbonyidiimidazole in anhydrous THF and the mixture is stirred at RT for 1 hour to activate the free acid moiety. Subsequently, the activated glutamic acid derivative is cannulated into a solution of Mg salt 49b and the reaction mixture obtained is stirred at RT for 16 hours. The mixture then is diluted with ethyl acetate and the organic solution is washed with 0.5 N HCl (at 0° C.) and brine, dried and evaporated. The residue obtained is resolved via silica chromatography with a 35-40% ethyl acetate in hexanes eluent system to yield diester 49c.

49B. To a stirred solution of tetrakis (triphenylphosphine) Pd (0) in dry DMF is added the diester in DMF. The mixture is stirred at RT for 3.5 hours. The DMF is evaporated under reduced pressure and the residue diluted with EtOAc. The EtOAc solution is washed with 0.5N 0° C. HCl, brine, dried and evaporated. The residue is chromatographed on silica gel using 15% to 20% EtOAc in hexane as eluent to afford the methyl ester intermediate.

The methyl ester intermediate is then diluted with THF and water, LiOH.OH₂O is added and the resulting mixture is stirred at RT for 25 hours, wherein the completion of the hydrolysis is monitored by TLC. The reaction mixture is concentrated under vacuum to remove a majority of the THF and further diluted with methylene chloride. The resulting solution is washed with 1 N HCl, dried with anhydrous Na₂SO₄ and concentrated under vacuum. To remove minor impurities and excess Boc₂O, the crude product is purified via flash chromatography using a solvent gradient from 100% hexane→100% EtOAc as the eluent. (2S)-N-Boc-amino-5-oxo-non-8-enoic acid 49d is obtained. For further details of the preceding amino acid synthesis may be found in T. Tsuda et al., J. Am. Chem. Soc., 1980, 102, 6381-6384 and WO 00/59929.

49C. Synthesis of Modified Cyclic Peptide Precursor Mesylate

The modified cyclic peptide precursor mesylate is prepared using the synthetic route detailed in Example 1 using (2S)-N-Boc-amino-5-oxo-non-8-enoic acid 49d in place of Boc-L-2-amino-8-nonenoic acid 1a followed by conversion to the corresponding mesylate via the method described in Example 2.

The title compound is prepared with the modified cyclic peptide precursor mesylate formed in 49C and 4,5-di(thiophen-3-yl)-2H-pyridazin-3-one by the Mitsunobu conditions elucidated in Example 2 followed by hydrolysis of the ethyl ester via the method set forth in Example 4.

### Example 50

Compound of Formula I, wherein A=tBOC, G=OH, L=—CH(CH₃)CH₂—, X═Y=Thiophen-3-yl, Z=Hydrogen, j=1, m=s=1, R³=Methyl, and R⁴=Hydrogen.

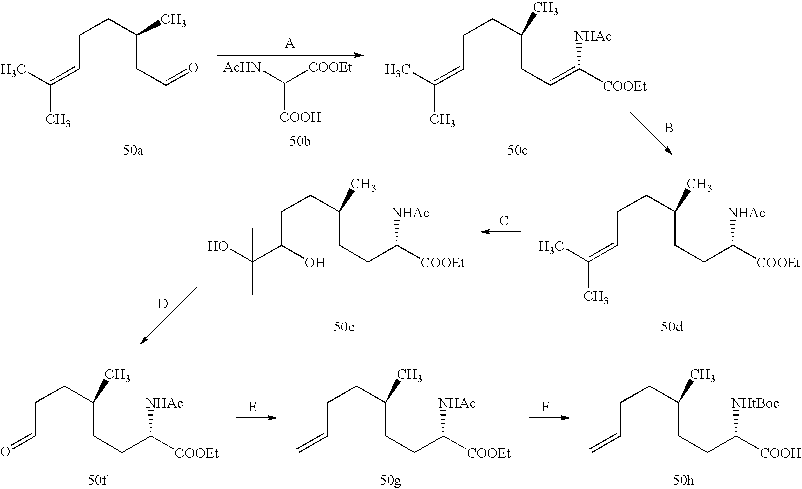

Synthesis of (2S, 5R)-N-Boc-2-amino-5-methyl-non-8-enoic Acid (50h).

50A. To solid ethyl 2-acetamidomalonate 50b is added (R)-(+)-citronellal 50a in a solution of pyridine over 1 min. The resulting solution is cooled in a 10° C. bath and acetic anhydride is added over 4 min. The resulting solution is stirred for 3 hours at RT and another portion of ethyl 2-acetamidomalonate 50a is added. The resulting mixture is stirred at RT for an additional 11 hours. Ice is then added and the solution is stirred for 1.5 hours, then the mixture is diluted with 250 ml water and extracted with two portions of ether. The organic phase is washed with 1 N HCl, sat. NaHCO₃, dried Na₂SO₄, concentrated and purified by flash chromatography (40% EtOAc/hexane) to afford compound 50c.

50B. To a degassed solution of 50c in dry ethanol is added (S,S)-Et-DUPHOS Rh(COD)OTf. The mixture is subjected to 30 psi of hydrogen and stirred on a Parr shaker for 2 hours. The resulting mixture is evaporated to dryness to obtain the crude compound 50d, which is used in the subsequent step without purification.

50C. Compound 50d is dissolved in a mixture of tBuOH/acetone/H₂O (1:1:1) and placed in an ice bath (0° C.). NMMO and OsO₄ is consecutively added and the reaction mixture is stirred at RT for 4 hours. A majority of the acetone is removed by evaporation under vacuum and then the mixture is extracted with ethyl acetate. The organic layer is further washed with water and brine, dried over anhydrous MgSO₄ and evaporated to dryness. The diol 50e is obtained in high purity after flash column chromatography using 1% ethanol in ethyl acetate as the eluent.

50D. To a solution of diol 50e in THF/H₂O (1:1) at 0° C., NalO₄ is added and the reaction mixture is stirred at RT for 3.5 hours. A majority of the THF solvent is subsequently removed by evaporation under vacuum and the remaining mixture is extracted with EtOAc. The combined organic layers are further washed with 5% aqueous citric acid solution, 5% aq. NaHCO₃ and brine, then the organic phase is dried over MgSO₄ and evaporated to dryness under vacuum. Aldehyde intermediate 50f is used in the following step in its crude form.

50E. To a solution of Ph₃PCH₃Br in anhydrous toluene, KHMDS is added forming a suspension which is stirred at RT for 30 min. under N₂. After stirring, the suspension is cooled to 0° C., a solution of aldehyde intermediate 50f in THF is added, the mixture is warmed to RT, and stirred for 1 hour. A majority of the THF is evaporated under vacuum, EtOAc is added to the mixture and the organic phase is washed with water, 5% aq. NaHCO₃ and brine. The organic phase is then dried over MgSO₄ and evaporated to dryness under vacuum. Pure compound 50 g is isolated after purification via flash chromatography on silica gel, using hexane:EtOAc (3:2) as the eluent.

50F. To a solution of crude 50 g in THF, Boc₂O, and DMAP is added and the reaction mixture is heated to reflux for 2.5 hours. Subsequently, a majority of the THF is evaporated, the crude mixture is diluted with methylene chloride and washed with 1 N HCl to remove DMAP. The organic layer is further extracted with saturated aq. NaHCO₃, dried with anyhrous Na₂SO₄ and concentrated under vacuum. The crude product is then diluted with THF and water, LiOH.H₂O is added and the resulting mixture is stirred at RT for 25 hours, wherein the completion of the hydrolysis is monitored by TLC. The reaction mixture is concentrated under vacuum to remove a majority of the THF and further diluted with methylene chloride. The resulting solution is washed with 1 N HCl, dried with anhydrous Na₂SO₄ and concentrated under vacuum. To remove minor impurities and excess Boc₂O, the crude product is purified via flash chromatography using a solvent gradient from 100% hexane→100% EtOAc as the eluent. (2S, 5R)-N-Boc-2-amino-5-methyl-non-8-enoic acid 50h is obtained. For further details of the preceding amino acid synthesis see WO 00/59929.

Synthesis of Modified Cyclic Peptide Precursor Mesylate

The modified cyclic peptide precursor mesylate is prepared using the synthetic route detailed in Example 1 using ((2S, 5R)-N-Boc-2-amino-5-methyl-non-8-enoic acid 50h in place of Boc-L-2-amino-8-nonenoic acid la followed by conversion to the corresponding mesylate via the method described in Example 2.

The title compound is prepared with the modified cyclic peptide precursor mesylate formed in 50G and 4,5-di(thiophen-3-yl)-2H-pyridazin-3-one by the Mitsunobu conditions elucidated in Example 2 followed by hydrolysis of the ethyl ester via the method set forth in Example 4.

### Example 51

Compound of Formula I, wherein A=tBOC, G=OH, L=—O—, X═Y=Thiophen-3-yl, Z=Hydrogen,=0, m=s=1, R³=methyl, and R⁴=Hydrogen.

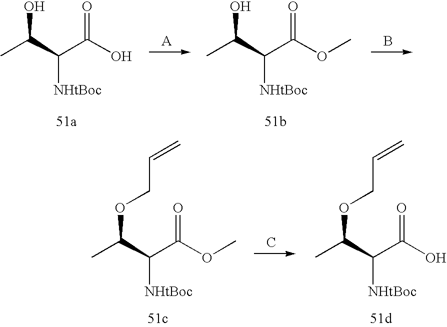

Synthesis of N-Boc-O-allyl-(L)-threonine (51d)

51A. Boc-(L)-threonine 51a is partially dissolved in methylene chloride/methanol at 0° C. A solution of diazomethane in diethyl ether is added until yellow, indicating the presence of diazomethane. Upon evaporation of the solvents, crude methyl ester 51 b is obtained.

51B. Intermediate 51 b is dissolved in anhydrous diethyl ether, Ag₂O is added and freshly activated 4 molecular sieves. Finally, allyl iodide is added to the reaction mixture and is stirred at reflux. Two additional portions of allyl iodide are added to the reaction mixture after a period of 20 hours and 30 hours and stirring is continued for a total of 36 hours. The mixture is then filtered through celite and purified by flash chromatography on silica gel, using EtOAc/hexane (1:4) as the eluent, to afford compound 51c.

51C. Compound 51c is dissolved in a mixture of THF/MeOH/H₂O (2:1:1) and LiOH.H₂O is added. The solution is stirred at RT for 2 hours, and is acidified with 1 N HCl to pH˜3 before the solvents are removed under vacuum. The resulting crude compound 51 d is obtained. For further details of the preceding synthesis see WO 00/59929, which is herein incorporated by reference in its entirety.

51 D. Synthesis of Modified Cyclic Peptide Precursor Mesylate

The modified cyclic peptide precursor mesylate is prepared using the synthetic route detailed in Example 1 using N-Boc-O-allyl-(L)-threonine 51d in place of Boc-L-2-amino-8-nonenoic acid 1a followed by conversion to the corresponding mesylate via the method described in Example 2.

The title compound is prepared with the modified cyclic peptide precursor mesylate formed in 51 D and 4,5-di(thiophen-3-yl)-2H-pyridazin-3-one by the Mitsunobu conditions elucidated in Example 2 followed by hydrolysis of the ethyl ester via the method set forth in Example 4.

### Example 52

Compound of Formula I, wherein A=tBOC, G=OH, L=—S—, X═Y=Thiophen-3-yl, Z=Hydrogen, j=0, m=s=1, R³=Methyl, and R⁴=Hydrogen.

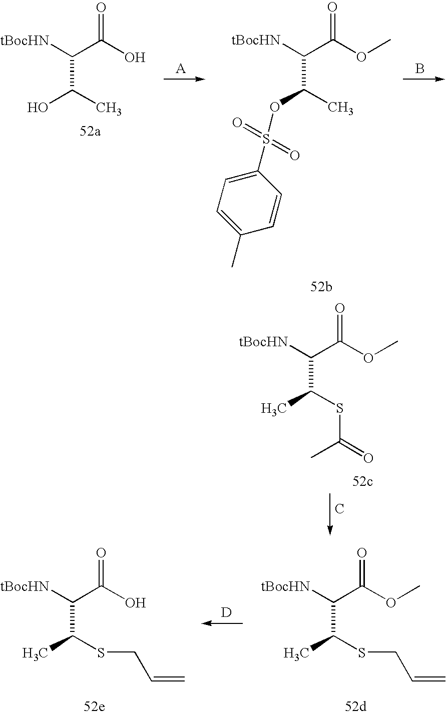

Synthesis of (2S, 3S)-N-Boc-2 Amino-3(Mercaptoallyl)butanoic Acid (52e).

52A. Compound 52a is dissolved in pyridine and the solution is cooled to 0° C. in an ice bath, tosyl chloride is added in small portions and the reaction mixture is partitioned between diethyl ether and H₂O. The ether layer is further washed with 0.2 N HCl and brine, dried over anhydrous MgSO₄, filtered and concentrated to dryness under vacuum. Purification of the crude material by flash chromatography on silica gel, using hexane/EtOAc (gradient from 8:2 to 7:3 ratio) as the eluent, leads to isolation of tosyl derivative 52b.

52B. To a solution of tosyl derivative 52b in anhydrous DMF, potassium thioacetate is added and the reaction mixture is stirred at RT for 24 hours. A majority of the DMF is then evaporated under vacuum and the remaining mixture is partitioned between EtOAc and H₂O. The aqueous layer is re-extracted with EtOAc, the combined organic layers are washed with brine, dried over anhydrous MgSO₄ and evaporated to dryness. Purification of the crude material by flash chromatography on silica gel using hexane/EtOAc (4:1 ratio) as the eluent, affords thioester 52c.

52C. To a solution of thioester 52c is H₂O/EtOH (3:5 ratio) and aqueous solution of 0.2M NaOH is added and the mixture is stirred at RT for 1.5 hours. Allyl iodide is then added and stirring is continued at RT for an additional 30 min. The reaction mixture is concentrated to half of its original volume and then extracted with EtOAc. The aqueous layer is acidified to pH ˜3 with cold, aqueous 0.5N HCl and re-extracted with EtOAc. The combined organic layers are washed with brine, dried over anhydrous MgSO₄ and evaporated to dryness under vacuum. The crude reaction mixture contains at least four products; all of the products are isolated after flash chromatography on silica gel, using hexane/EtOAc (gradient from 9:1 to 3:1). The desired product 52d is the least polar compound.

52D. A solution of compound 52d in MeOH/H₂O (3:1) is mixed with aqueous NaOH (0.3 N) for 24 hours at RT and for 1 hour at 40° C. The reaction mixture is acidified with cold aqueous 0.5 N HCl, the MeOH is removed under vacuum and the remaining aqueous mixture is extracted with EtOAc. The organic phase is dried over MgSO₄ and evaporated to dryness in order to obtain compound 52e. For further details of the synthesis of amino acid 52e, see WO 00/59929, which is herein incorporated by reference in its entirety.

52E. Synthesis of Modified Cyclic Peptide Precursor Mesylate

The modified cyclic peptide precursor mesylate is prepared using the synthetic route detailed in Example 1 using (2S, 3S)-N-Boc-2 amino-3(mercaptoallyl)butanoic acid 51e in place of Boc-L-2-amino-8-nonenoic acid 1a followed by conversion to the corresponding mesylate via the method described in Example 2.

The title compound is prepared with the modified cyclic peptide precursor mesylate formed in 52E and 4,5-di(thiophen-3-yl)-2H-pyridazin-3-one by the Mitsunobu conditions elucidated in Example 2 followed by hydrolysis of the ethyl ester via the method set forth in Example 4.

### Example 53

Compound of Formula I, wherein A=tBOC, G=OH, L=—S(O)—, X═Y=Thiophen-3-yl, Z=Hydrogen, j=2, m=s=1, R³=Methyl, and R⁴=Hydrogen.

Formation of Modified Amino Acid

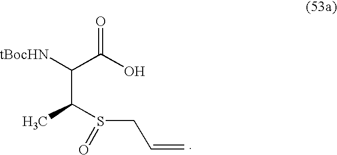

53A. The modified amino acid is prepared by dissolving sodium metaperiodate (1.1 eq.) in water and cooled to 0° C. in an ice bath followed by adding dropwise a solution of compound 52d in dioxane. The resulting reaction mixture is stirred for one hour at 0° C. and 4 hours at 40° C. The reaction mixture is concentrated, water is added, and the mixture is extracted with methylene chloride twice. The combined organic layers are washed with water, brine, dried with anhydrous MgSO₄ and concentrated in vacuo. The methyl ester is then reduced via the method set forth in Example 52D to arrive upon the modified amino acid 53a. For further details concerning the sulfur oxidation reaction, see S. A. Burrage et al., Tett. Lett., 1998, 39, 2831-2834, which is herein incorporated by reference in its entirety.

53B. Synthesis of Modified Cyclic Peptide Precursor Mesylate

The modified cyclic peptide precursor mesylate is prepared using the synthetic route detailed in Example 1 using the modified amino acid 53a in place of Boc-L-2-amino-8-nonenoic acid la followed by conversion to the corresponding mesylate via the method described in Example 2.

The title compound is prepared with the modified cyclic peptide precursor mesylate formed in 53B and 4,5-di(thiophen-3-yl)-2H-pyridazin-3-one by the Mitsunobu conditions elucidated in Example 2 followed by hydrolysis of the ethyl ester via the method set forth in Example 4.

### Example 54

Compound of Formula I, wherein A=tBOC, G=OH, L=—S(O)₂ X═Y=Thiophen-3-yl, Z=Hydrogen, j=2, m=s=1, R³=Methyl, and R⁴=Hydrogen.

Formation of Modified Amino Acid

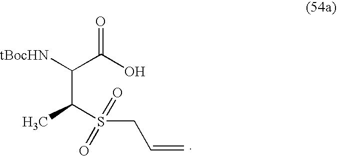

54A. The modified amino acid is prepared by dissolving sodium metaperiodate (1.1 eq.) in water and cooled to 0° C. in an ice bath followed by adding dropwise a solution of compound 52d in dioxane. The resulting reaction mixture is stirred for one hour at 0° C. and 4 hours at 40° C. The reaction mixture is concentrated, water is added, and the mixture is extracted with methylene chloride twice. The combined organic layers are washed with water, brine, dried with anhydrous MgSO₄ and concentrated in vacuo. The methyl ester is then reduced via the method set forth in Example 52D to arrive upon the modified amino acid 54a. For further details concerning the sulfur oxidation reaction, see S. A. Burrage et al., Tett. Lett., 1998, 39, 2831-2834.

54B. Synthesis of Modified Cyclic Peptide Precursor Mesylate

The modified cyclic peptide precursor mesylate is prepared using the synthetic route detailed in Example 1 using the modified amino acid 48a in place of Boc-L-2-amino-8-nonenoic acid la followed by conversion to the corresponding mesylate via the method described in Example 2.

The title compound is prepared with the modified cyclic peptide precursor mesylate formed in 54B and 4,5-di(thiophen-3-yl)-2H-pyridazin-3-one by the Mitsunobu conditions elucidated in Example 2 followed by hydrolysis of the ethyl ester via the method set forth in Example 4.

### Example 55

Compound of Formula I, wherein A=tBOC, G=OH, L=—SCH₂CH₂—, X=Y=Thiophen-3-yl, Z=Hydrogen, j=0, m=s=1, and R³═R⁴=CH₃.

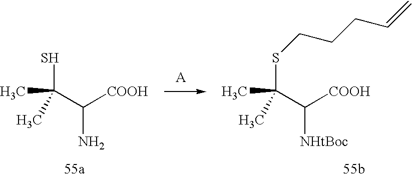

55A. Synthesis of (S)-N-Boc-2-amino-3-methyl-3(1-mercapto-4-butenyl)butanoic acid (55b)

L-Penicillamine 55a is dissolved in DMF/DMSO (5:1), subsequently, 4-bromopentene and CsOH.H₂O are added to the mixture and stirring is continued for an additional 12 hours. The DMF is subsequently removed in vacuo, the remaining mixture is diluted with 0.5 N HCl (at 0° C.) to adjust the pH to ˜4-5 and then extracted with 2 portions of EtOAc. The organic phase is washed with brine (2×), dried over MgSO₄ and evaporated to dryness to afford the crude carboxylic acid 55a.

55B. Synthesis of Modified Cyclic Peptide Precursor Mesylate

The modified cyclic peptide precursor mesylate is prepared using the synthetic route detailed in Example 1 using the modified amino acid 55a in place of Boc-L-2-amino-8-nonenoic acid la followed by conversion to the corresponding mesylate via the method described in Example 2.

The title compound is prepared with the modified cyclic peptide precursor mesylate formed in 55B and 4,5-di(thiophen-3-yl)-2H-pyridazin-3-one by the Mitsunobu conditions elucidated in Example 2 followed by hydrolysis of the ethyl ester via the method set forth in Example 4.

### Example 56

Compound of Formula I, wherein A=tBOC, G=OH, L=CF₂CH₂, X═Y=Thiophen-3-yl, Z=Hydrogen, j=1, m=s=1, and R³═R⁴=Hydrogen.

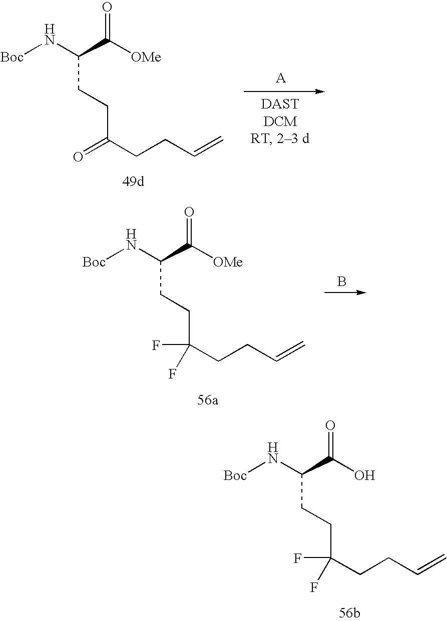

Synthesis of (2S)-N-Boc-amino-5-difluoro-non-8-enoic Acid (56b).

56A. To a solution of the ketone compound 56d (0.30 g, 1 mmol) in 5 ml DCM, DAST (Diethylaminosulfurtrifluoride, 0.2 g, 1.2 eq) is added. The reaction is kept at RT over a period of 2-3 days. The solvent is evaporated and the residue is purified by silica gel flash chromatography using different ratios of hexanes:EtOAc as eluent (9:1→5:1→3:1→1:1), providing the isolated methyl ester 56a. For further details concerning the preceding synthesis, see Tius, Marcus A et al., Tetrahedron, 1993, 49, 16; 3291-3304, which is herein incorporated by reference in its entirety.

56B. Methyl ester 56a is dissolved in THF/MeOH/H₂O (2:1:1) and LiOH.OH₂O is added. The solution is stirred at RT for 2 hours, and is then acidified with 1 N HCl to pH˜3 before the solvents are removed in vacuo to afford the crude (2S)-N-Boc-amino-5-difluoro-non-8-enoic acid 56b.

56C. Synthesis of Modified Cyclic Peptide Precursor Mesylate

The modified cyclic peptide precursor mesylate is prepared using the synthetic route detailed in Example 1 using crude (2S)-N-Boc-amino-5-difluoro-non-8-enoic acid 56b in place of Boc-L-2-amino-8-nonenoic acid la followed by conversion to the corresponding mesylate via the method described in Example 2.

The title compound is prepared with the modified cyclic peptide precursor mesylate formed in 56C and 4,5-di(thiophen-3-yl)-2H-pyridazin-3-one by the Mitsunobu conditions elucidated in Example 2 followed by hydrolysis of the ethyl ester via the method set forth in Example 4.

### Example 57

Compound of Formula I, wherein A=tBOC, G=OH, L=—CHFCH₂—, X=Y=Thiophen-3-yl, Z=Hydrogen, j=1, m=S=1, and R³═R⁴=Hydrogen.

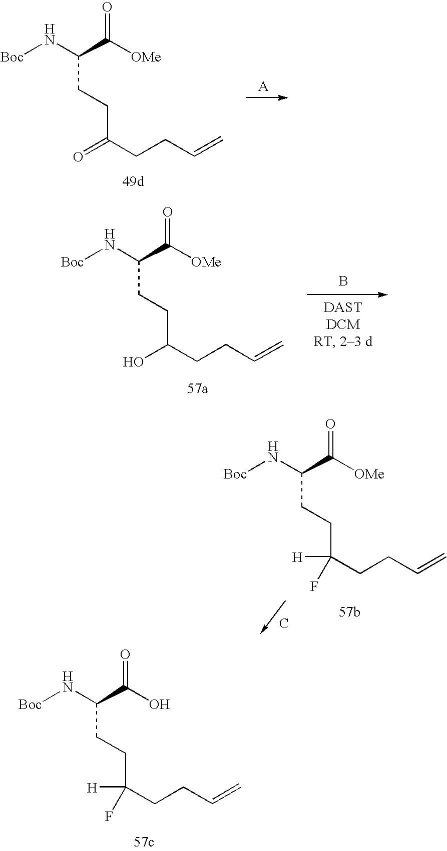

Synthesis of (2S)-N-Boc-amino-5-fluoro-non-8-enoic Acid (57c).

57A. To a solution of the ketone compound 49d in 5 ml methanol, NaBH₄ (2.2 eq) is added. The reaction mixture is stirred at RT over a period of 2-6 hours, and then quenched by 1 M ammonium chloride and extracted with EtOAc (30 ml). The solvent is evaporated and the crude hydroxy compound 57a is obtained.

57B. The hydroxy compound 57a is dissolved in 5 ml DCM to which DAST (0.2 g, 1.2 eq) is added and stirred at −45° C. for 1 hour. The reaction mixture is then warmed to RT and stirred over a period of 2-3 days. The solvent is evaporated and the residue is purified by silica gel flash chromatography using different ratios of hexanes:EtOAc as eluent (9:1→5:1→3:1→1:1), providing the isolated monofluoro compound methyl ester 57b. For further details concerning the preceding reaction, see Buist, Peter H et al., Tetrahedron Lett., 1987, 28, 3891-3894, which is herein incorporated by reference in its entirety.

57C. Methyl ester 57b is dissolved in THF/MeOH/H₂O (2:1:1) and LiOH.H₂O is added. The solution is stirred at RT for 2hours, and is then acidified with 1 N HCl to pH˜3 before the solvents are removed in vacuo to afford the crude (2S)-N-Boc-amino-5-difluoro-non-8-enoic acid 57c.

57D. Synthesis of Modified Cyclic Peptide Precursor Mesylate

The modified cyclic peptide precursor mesylate is prepared using the synthetic route detailed in Example 1 using crude (2S)-N-Boc-amino-5-monofluoro-non-8-enoic acid 57b in place of Boc-L-2-amino-8-nonenoic acid 1a followed by conversion to the corresponding mesylate via the method described in Example 2.

The title compound is prepared with the modified cyclic peptide precursor mesylate formed in 57C and 4,5-di(thiophen-3-yl)-2H-pyridazin-3-one by the Mitsunobu conditions elucidated in Example 2 followed by hydrolysis of the ethyl ester via the method set forth in Example 4.

### Example 58

Compound of Formula II, wherein A=tBOC, G=OH, L=Absent, X═Y=Thiophen-3-yl, Z=Hydrogen, j=3, m=s=1, and R³═R⁴=Hydrogen.

58A. The saturated cyclic peptide precursor mesylate is prepared by catalytic reduction of the mesylate cyclic peptide precursor of Example 1 with Pd/C in MeOH in the presence of H₂.

The title compound is prepared with the saturated cyclic peptide precursor mesylate formed in 58A and 4,5-di(thiophen-3-yl)-2H-pyridazin-3-one by the Mitsunobu conditions elucidated in Example 2 followed by hydrolysis of the ethyl ester via the method set forth in Example 4.

The compounds of the present invention exhibit potent inhibitory properties against the HCV NS3 protease. The following examples elucidate assays in which the compounds of the present invention are tested for anti-HCV effects.

### Example 59

NS3/NS4a Protease Enzyme Assay

HCV protease activity and inhibition is assayed using an internally quenched fluorogenic substrate. A DABCYL and an EDANS group are attached to opposite ends of a short peptide. Quenching of the EDANS fluorescence by the DABCYL group is relieved upon proteolytic cleavage. Fluorescence was measured with a Molecular Devices Fluoromax (or equivalent) using an excitation wavelength of 355 nm and an emission wavelength of 485 nm.

The assay is run in Corning white half-area 96-well plates (VWR 29444-312 [Corning 3693]) with full-length NS3 HCV protease 1b tethered with NS4A cofactor (final enzyme concentration 1 to 15 nM). The assay buffer is complemented with 10 μM NS4A cofactor Pep 4A (Anaspec 25336 or in-house, MW 1424.8). RET S1 (Ac-Asp-Glu-Asp(EDANS)-Glu-Glu-Abu-[COO]Ala-Ser-Lys-(DABCYL)-NH₂, AnaSpec 22991, MW 1548.6) is used as the fluorogenic peptide substrate. The assay buffer contained 50 mM Hepes at pH 7.5, 30 mM NaCl and 10 mM BME. The enzyme reaction is followed over a 30 minutes time course at room temperature in the absence and presence of inhibitors.

The peptide inhibitors HCV Inh 1 (Anaspec 25345, MW 796.8) Ac-Asp-Glu-Met-Glu-Glu-Cys-OH, [−20° C.] and HCV Inh 2 (Anaspec 25346, MW 913.1) Ac-Asp-Glu-Dif-Cha-Cys-OH, were used as reference compounds.

IC50 values were calculated using XLFit in ActivityBase (IDBS) using equation 205: y=A+((B−A)/(1+((C/x)\hat{ }D))).

### Example 60

Cell-Based Replicon Assay

Quantification of HCV Replicon RNA in Cell Lines (HCV Cell Based Assay) Cell lines, including Huh-11-7 or Huh 9-13, harboring HCV replicons (Lohmann, et al Science 285:110-113, 1999) are seeded at 5×10³ cells/well in 96 well plates and fed media containing DMEM (high glucose), 10% fetal calf serum, penicillin-streptomycin and non-essential amino acids. Cells are incubated in a 5% CO₂ incubator at 37° C. At the end of the incubation period, total RNA is extracted and purified from cells using Qiagen Rneasy 96 Kit (Catalog No. 74182). To amplify the HCV RNA so that sufficient material can be detected by an HCV specific probe (below), primers specific for HCV (below) mediate both the reverse transcription of the HCV RNA and the amplification of the cDNA by polymerase chain reaction (PCR) using the TaqMan One-Step RT-PCR Master Mix Kit (Applied Biosystems catalog no. 4309169). The nucleotide sequences of the RT-PCR primers, which are located in the NS5B region of the HCV genome, are the following:

HCV Forward primer “RBNS5bfor”
5′GCTGCGGCCTGTCGAGCT:
HCV Reverse primer “RBNS5Brev”:
5′CAAGGTCGTCTCCGCATAC

Detection of the RT-PCR product was accomplished using the Applied Biosystems (ABI) Prism 7700 Sequence Detection System (SDS) that detects the fluorescence that is emitted when the probe, which is labeled with a fluorescence reporter dye and a quencher dye, is processed during the PCR reaction. The increase in the amount of fluorescence is measured during each cycle of PCR and reflects the increasing amount of RT-PCR product. Specifically, quantification is based on the threshold cycle, where the amplification plot crosses a defined fluorescence threshold. Comparison of the threshold cycles of the sample with a known standard provides a highly sensitive measure of relative template concentration in different samples (ABI User Bulletin #2 Dec. 11, 1997). The data is analyzed using the ABI SDS program version 1.7. The relative template concentration can be converted to RNA copy numbers by employing a standard curve of HCV RNA standards with known copy number (ABI User Bulletin #2 Dec. 11, 1997).

The RT-PCR product was detected using the following labeled probe:

5° FAM-CGAAGCTCCAGGACTGCACGATGCT-TAMRA

FAM=Fluorescence reporter dye.

TAMRA:=Quencher dye.

The RT reaction is performed at 48° C. for 30 minutes followed by PCR. Thermal cycler parameters used for the PCR reaction on the ABI Prism 7700 Sequence Detection System were: one cycle at 95° C., 10 minutes followed by 35 cycles each of which included one incubation at 95° C. for 15 seconds and a second incubation for 60° C. for 1 minute.

To normalize the data to an internal control molecule within the cellular RNA, RT-PCR is performed on the cellular messenger RNA glyceraldehydes-3-phosphate dehydrogenase (GAPDH). The GAPDH copy number is very stable in the cell lines used. GAPDH RT-PCR is performed on the same exact RNA sample from which the HCV copy number is determined. The GAPDH primers and probes, as well as the standards with which to determine copy number, are contained in the ABI Pre-Developed TaqMan Assay Kit (catalog no. 4310884E). The ratio of HCV/GAPDH RNA is used to calculate the activity of compounds evaluated for inhibition of HCV RNA replication.

Activity of Compounds as Inhibitors of HCV Replication (Cell Based Assay) in Replicon Containing Huh-7 Cell Lines

The effect of a specific anti-viral compound on HCV replicon RNA levels in Huh-11-7 or 9-13 cells was determined by comparing the amount of HCV RNA normalized to GAPDH (e.g. the ratio of HCV/GAPDH) in the cells exposed to compound versus cells exposed to the 0% inhibition and the 100% inhibition controls. Specifically, cells were seeded at 5×10³ cells/well in a 96 well plate and were incubated either with: 1) media containing 1% DMSO (0% inhibition control), 2) 100 international units, IU/mi Interferon-alpha 2b in media/1% DMSO or 3) media/1% DMSO containing a fixed concentration of compound. 96 well plates as described above were then incubated at 37° C. for 3 days (primary screening assay) or 4 days (IC50 determination). Percent inhibition was defined as:

% Inhibition=[100-((S-C2)/C₁-C₂))]×100

where

S=the ratio of HCV RNA copy number/GAPDH RNA copy number in the sample;

C1=the ratio of HCV RNA copy number/GAPDH RNA copy number in the 0% inhibition control (media/1% DMSO); and

C2=the ratio of HCV RNA copy number/GAPDH RNA copy number in the 100% inhibition control (100 IU/ml Interferon-alpha 2b).

The dose-response curve of the inhibitor was generated by adding compound in serial, three-fold dilutions over three logs to wells starting with the highest concentration of a specific compound at 10 uM and ending with the lowest concentration of 0.01 uM. Further dilution series (1 uM to 0.001 uM for example) was performed if the IC50 value was not in the linear range of the curve. IC50 was determined based on the IDBS Activity Base program using Microsoft Excel “XL Fit” in which A=100% inhibition value (100 U/ml Interferon-alpha 2b), B=0% inhibition control value (media/1% DMSO) and C=midpoint of the curve as defined as C=(B−A/2)+A. A, B and C values are expressed as the ratio of HCV RNA/GAPDH RNA as determined for each sample in each well of a 96 well plate as described above. For each plate the average of 4 wells were used to define the 100% and 0% inhibition values.

Although the invention has been described with respect to various preferred embodiments, it is not intended to be limited thereto, but rather those skilled in the art will recognize that variations and modifications may be made therein which are within the spirit of the invention and the scope of the appended claims.

What is claimed:

1. A compound of Formula I: 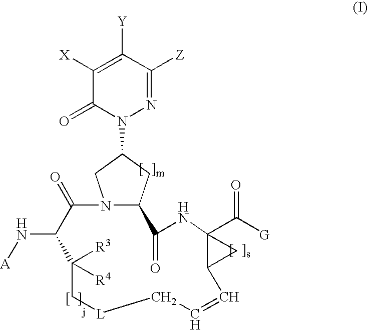
wherein
A is hydrogen, —(C═O)—R², —(C═O)—O—R¹, —C(═O)—NH—R², —C(═S)—NH—R², or —S(O)₂—R²;
G is —OH, —O—(C₁-C₁₂ alkyl), —NHS(O)₂—R¹, —(C═O)—R², —(C═O)—O—R¹, or —(C═O)—NH—R²;
L is —S—, —SCH₂—, —SCH₂CH₂—, —S(O)₂—, —S(O)₂CH₂CH₂—, —S(O)—, —S(O)CH₂CH₂—, —O—, —OCH₂—, —OCH₂CH₂—, —(C═O)—CH₂—, —CH(CH₃)CH₂—, —CFHCH₂— or —CF₂CH₂—;
X, Y, and Z are independently selected from the group consisting of hydrogen, N₃, halogen, C₁-C₆ alkyl, C₃-C₁₂ cycloalkyl, alkylamino, dialkylamino, C₁-C₆ alkynyl, substituted alkynyl, aryl, substituted aryl, —S-aryl, —S-substituted aryl, —O-aryl, —O-substituted aryl, NH-aryl, NH-substituted aryl, diarylamino, diheteroarylamino, arylalkyl, substituted arylalkyl, heteroaryl, substituted heteroaryl, —S-heteroaryl, —S-substituted heteroaryl, —O-heteroaryl, —O-substituted heteroaryl, —NH-heteroaryl, —NH-substituted heteroaryl, heteroarylalkyl, substituted heteroarylalkyl, heterocycloalkyl, and substituted heterocycloalkyl; or, in the alternative, X and Y or Y and Z taken together with the carbon atoms to which they are attached form an aryl, substituted aryl, heteroaryl, or substituted heteroaryl cyclic moiety;
j=0, 1, 2, 3, or 4;
m=0, 1, or 2;
s=0, 1 or 2;
R¹ is hydrogen, C₁-C₆ alkyl, C3-C₁₂ cycloalkyl, substituted C₃-C₁₂ cycloalkyl, aryl, substituted aryl, arylalkyl, substituted arylalkyl, heteroaryl, substituted heteroaryl, heteroarylalkyl, substituted heteroarylalkyl, heterocycloalkyl, or substituted heterocycloalkyl;
R² is hydrogen, C₁-C₆ alkyl, C₃-C₁₂ cycloalkyl, substituted C₃-C₁₂ cycloalkyl, alkylamino, dialkyl amino, arylamino, diarylamino, aryl, substituted aryl, arylalkyl, substituted arylalkyl, heteroaryl, substituted heteroaryl, heteroarylalkyl, substituted heteroarylalkyl, heterocycloalkyl, or substituted heterocycloalkyl; and
R³ and R⁴ are each independently hydrogen or methyl.

2. A compound according to claim 1, wherein:
A is —(C═O)—O—R¹;
G is hydroxyl;
L is absent;
j=3;
m=s=1; and
R³ and R⁴ are hydrogen.

3. A compound according to claim 1, wherein:
A is —(C═O)—O-tert-butyl;
G is hydroxyl;
L is absent;
j=3;
m=s=1; and
R³ and R⁴ are hydrogen.

4. A compound according to claim 1 which is selected from the group consisting of:
Compound of Formula I, wherein A=tBOC, G=OEt, L=absent, X═Y=bromo, Z=hydrogen, j=3, m=s=1, and R³═R⁴=hydrogen;
Compound of Formula I, wherein A=tBOC, G=OEt, L=absent, X═Y=thiophen-3-yl, Z=hydrogen, j=3, m=s=1, and R³═R⁴=hydrogen;
Compound of Formula I, wherein A=tBOC, G=OH, L=absent, X═Y=thiophen-3-yl, Z=hydrogen, j=3, m=s=1, and R³═R⁴=hydrogen;
Compound of Formula I, wherein A=tBOC, G=OH, L=absent, X═Y=phenyl, Z=hydrogen, j=3, m=s=1, and R³═R⁴=hydrogen;
Compound of Formula I, wherein A=tBOC, G=OH, L=absent, X═Y=4-(N,N-dimethylamino)phenyl, Z=hydrogen, j=3, m=s=1, and R³═R⁴=hydrogen;
Compound of Formula I, wherein A=tBOC, G=OH, L=absent, X═Y=4-(trifluoromethoxy)phenyl, Z=hydrogen, j=3, m=s=1, and R³═R⁴=hydrogen;
Compound of Formula I, wherein A=tBOC, G=OH, L=absent, X═Y=4-(methanesulfonyl)phenyl, Z=hydrogen, j=3, m=s=1, and R³═R⁴=hydrogen;
Compound of Formula I, wherein A=tBOC, G=OH, L=absent, X═Y=4-(cyano)phenyl, Z=hydrogen, j=3, m=s=1, and R³═R⁴=hydrogen;
Compound of Formula I, wherein A=tBOC, G=OH, L=absent, X═Y=3-pyridyl, Z=hydrogen, j=3, m=s=1, and R³═R⁴=hydrogen;
Compound of Formula I, wherein A=tBOC, G=OH, L=absent, X═Y=4-(morpholin-4-yl-methanonyl)phenyl, Z=hydrogen, j=3, m=s=1, and R³═R⁴=hydrogen;
Compound of Formula I, wherein A=tBOC, G=OH, L=absent, X═Y=bromo, Z=hydrogen, j=3, m=s=1, and R³═R⁴=hydrogen;
Compound of Formula I, wherein A=tBOC, G=OH, L=absent, X and Y taken together=phenyl, Z=4-methoxyphenyl, j=3, m=s=1, and R³═R⁴=hydrogen;
Compound of Formula I, wherein A=tBOC, G=OH, L=absent, X and Y taken together=phenyl, Z=4-chlorophenyl, j=3, m=s=1, and R³═R⁴=hydrogen;
Compound of Formula I, wherein A=tBOC, G=OH, L=absent, X=4-fluorophenyl, Y=hydrogen, Z=phenyl, j=3, m=s=1, and R³═R⁴=hydrogen;
Compound of Formula I, wherein A=tBOC, G=OH, L=absent, X=hydrogen, Y=1-piperidyl, Z=phenyl, j=3, m=s=1, and R³═R⁴=hydrogen;
Compound of Formula I, wherein A=tBOC, G=OEt, L=absent, X=hydrogen, Y=bromo, Z=phenyl, j=3, m=s=1, and R³═R⁴=hydrogen;
Compound of Formula I, wherein A=tBOC, G=OH, L=absent, X=hydrogen, Y=thiophen-3-yl, Z=phenyl, j=3, m=s=1, and R³═R⁴=hydrogen;
Compound of Formula I, wherein A=tBOC, G=OEt, L=absent, X=bromo, Y=pyrrolid-1-yl, Z=hydrogen, j=3, m=s=1, and R³═R⁴=hydrogen;
Compound of Formula I, wherein A=tBOC, G=OH, L=absent, X=thiophen-3-yl, Y=pyrrolid-1-yl, Z=hydrogen, j=3, m=s=1, and R³═R⁴=hydrogen;
Compound of Formula I, wherein A=tBOC, G=OEt, L=absent, X=bromo, Y=azido, Z=hydrogen, j=3, m=s=1, and R³═R⁴=hydrogen;
Compound of Formula I, wherein A=tBOC, G=OEt, L=absent, X=thiophen-3-yl, Y=azido, Z=hydrogen, j=3, m=s=1, and R³═R⁴=hydrogen;
Compound of Formula I, wherein A=tBOC, G=OH, L=absent, X=thiophen-3-yl, Y=azido, Z=hydrogen, j=3, m=s=1, and R³═R⁴=hydrogen;
Compound of Formula I, wherein A=tBOC, G=OH, L=absent, X=thiophen-3-yl, Y=tetrazol-2-yl, Z=hydrogen, j=3, m=s=1, and R³═R⁴=hydrogen;
Compound of Formula I, wherein A=tBOC, G=OH, L=absent, X═Y=mercapto-2-pryrimidine, Z=hydrogen, j=3, m=s=1, and R³═R⁴=hydrogen;
Compound of Formula I, wherein A=tBOC, G=OH, L=absent, X=bromo, Y=mercapto-2-pryrimidine, Z=hydrogen, j=3, m=s=1, and R³═R⁴=hydrogen;
Compound of Formula I, wherein A=tBOC, G=OH, L=absent, X=thiophen-3-yl, Y=mercapto-2-pryrimidine, Z=hydrogen, j=3, m=s=1, and R³═R⁴=hydrogen;
Compound of Formula I, wherein A=tBOC, G=OH, L=absent, X═Y=thiazol-2-yl, Z=hydrogen, j=3, m=s=1, and R³═R⁴=hydrogen;
Compound of Formula I, wherein A=tBOC, G=OH, L=absent, X═Y=imidazol-1-yl, Z=hydrogen, j=3, m=s=1, and R³═R⁴=hydrogen;
Compound of Formula I, wherein A=tBOC, G=OH, L=absent, X=2-(cyclopropylamino)-thiazol-4-yl, Y=4-methoxyphenyl, Z=hydrogen, j=3, m=s=1, and R³═R⁴=hydrogen; and
Compound of Formula I, wherein A=tBOC, G=OH, L=absent, X and Y taken together=6-methoxy-isoquinolinyl, Z=hydrogen, j=3, m=s=1, and R³═R⁴=hydrogen.

5. A compound according to claim 1 which is selected from the group consisting of:
Compound of Formula I, wherein A=—(C═O)—O—R¹, wherein R¹=cyclopentyl, G=OH, L=absent, X=thiophen-3-yl, Y=thiophen-3-yl, Z=hydrogen, j=3, m=s=1, and R³═R⁴=hydrogen;
Compound of Formula I, wherein A=—(C═O)—O—R¹, wherein R¹=cyclobutyl, G=OH, L=absent, X=thiophen-3-yl, Y=thiophen-3-yl, Z=hydrogen, j=3, m=s=1, and R³═R⁴=hydrogen;
Compound of Formula I, wherein A=—(C═O)—O—R¹, wherein R¹=cyclohexyl, G=OH, L=absent, X=thiophen-3-yl, Y=thiophen-3-yl, Z=hydrogen, j=3, m=s=1, and R³═R⁴=hydrogen;
Compound of Formula I, wherein A=—(C═O)—O—R¹, wherein R¹= 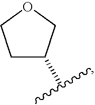
G=OH, L=absent X=thiophen-3-yl, Y=thiophen-3-yl, Z=hydrogen, j=3, m=s=1, and R³═R⁴=hydrogen;
Compound of Formula I, wherein A=—(C═O)—O—R¹, wherein R¹= 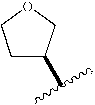
G=OH, L=absent, X=thiophen-3-yl, Y=thiophen-3-yl, Z=hydrogen, j=3, m=s=1, and R³═R⁴=hydrogen; and
Compound of Formula I, wherein A=—(C═O)—O—R¹, wherein R¹= 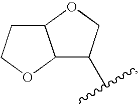
G=OH, L=absent, X=thiophen-3-yl, Y=thiophen-3-yl, Z=hydrogen, j=3, m=s=1, and R³═R⁴=hydrogen.

6. A compound according to claim 1 which is selected from the group consisting of:
Compound of Formula I, wherein A=tBOC, G=OH, L=—(C═O)CH₂—, X=thiophen-3-yl, Y=thiophen-3-yl, Z=hydrogen, j=1, m=s=1, and R³═R⁴=hydrogen;
Compound of Formula I, wherein A=tBOC, G=OH, L=—CH(CH₃)CH₂—, X=thiophen-3-yl, Y=thiophen-3-yl, Z=hydrogen, j=1, m=s=1, R³=methyl, and R⁴=hydrogen;
Compound of Formula I, wherein A=tBOC, G=OH, L=—O—, X=thiophen-3-yl, Y=thiophen-3-yl, Z=hydrogen, j=0, m=s=1, R³=methyl, and R⁴=hydrogen;
Compound of Formula I, wherein A=tBOC, G=OH, L=—S—, X=thiophen-3-yl, Y=thiophen-3-yl, Z=hydrogen, j=0, m=s=1, R³=methyl, and R⁴=hydrogen;
Compound of Formula I, wherein A=tBOC, G=OH, L=—S(O)—, X=thiophen-3-yl, Y=thiophen-3-yl, Z=hydrogen, j=2, m=s=1, R³=methyl, and R⁴=hydrogen;
Compound of Formula I, wherein A=tBOC, G=OH, L=—S(O)₂—, X=thiophen-3-yl, Y=thiophen-3-yl, Z=hydrogen, j=2, m=s=1, R³=methyl, and R⁴=hydrogen;
Compound of Formula I, wherein A=tBOC, G=OH, L=—SCH₂CH₂—, X=thiophen-3-yl, Y=thiophen-3-yl, Z=hydrogen, j=0, m=s=1, and R³═R⁴=CH₃;
Compound of Formula I, wherein A=tBOC, G=OH, L=—CF₂CH₂—, X=thiophen-3-yl, Y=thiophen-3-yl, Z=hydrogen, j=1, m=s=1, and R³═R⁴=hydrogen; and
Compound of Formula I, wherein A=tBOC, G=OH, L=—CFHCH₂—, X=thiophen-3-yl, Y=thiophen-3-yl, Z=hydrogen, j=1, m=s=1, and R³═R⁴=hydrogen.

7. A compound according to claim 1 which is selected from the group consisting of:
Compound of Formula I, wherein A=—(C═O)—O—R¹, R¹=cyclopentyl, G=—O-phenethyl, L=absent, X=thiophen-3-yl, Y=thiophen-3-yl, Z=hydrogen, j=3, m=s=1, and R³=R⁴=hydrogen;
Compound of Formula I, wherein A=—(C═O)—O—R¹, R¹=cyclopentyl, G=—NH-phenethyl, L=absent, X=thiophen-3-yl, Y=thiophen-3-yl, Z=hydrogen, j=3, m=s=1, and R³═R⁴=hydrogen;
Compound of Formula I, wherein A=—(C═O)—O—R¹, R¹=cyclopentyl, G=—NHS(O) ₂-phenethyl, L=absent, X=thiophen-3-yl, Y=thiophen-3-yl, Z=hydrogen, j=3, m=s=1, and R³═R⁴=hydrogen;
Compound of Formula I, wherein A=—(C═O)—O—R¹, R¹=cyclopentyl, G=—(C═O)—OH, L=absent, X=thiophen-3-yl, Y=thiophen-3-yl, Z=hydrogen, j=3, m=s=1, and and R³═R⁴=hydrogen;
Compound of Formula I, wherein A=—(C═O)—O—R¹, R¹=cyclopentyl, G=—(C═O)—O-phenethyl, L=absent, X=thiophen-3-yl, Y=thiophen-3-yl, Z=hydrogen, j=3, m=s=1, and R³═R⁴=hydrogen;
Compound of Formula I, wherein A=—(C═O)—O—R¹, R¹=cyclopentyl, G=—(C═O)—NH-phenethyl, L=absent, X=thiophen-3-yl, Y=thiophen-3-yl, Z=hydrogen, j=3, m=s=1, and R³═R⁴=hydrogen; and
Compound of Formula I, wherein A=—(C═O)—O—R¹, R¹=cyclopentyl, G=—(C═O)—NH—S(O)₂-benzyl, L=absent, X=thiophen-3-yl, Y=thiophen-3-yl, Z=hydrogen, j=3, m=s=1, and R³═R⁴=hydrogen.

8. A compound of Formula II: 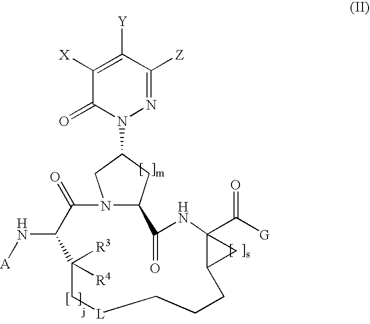
wherein
A is hydrogen, —(C═O)—R², —(C═O)—O—R¹, —C(═O)—NH—R², —C(═S)—NH—R², or —S(O)₂—R²;
G is —OH, —O—(C₁-C₁₂ alkyl), —NHS(O)₂—R¹, —(C═O)—R²—(C═O)—O—R¹, or —(C═O)—NH—R²;
L is absent, —S—, —SCH₂—, —SCH₂CH₂—, —S(O)₂—, —S(O)₂CH₂CH₂—, —S(O)—, —S(O)CH₂CH₂—, —O—, —OCH₂—, —OCH₂CH₂—, —(C═O)—CH₂—, —CH(CH₃)CH₂—, —CFHCH₂—, or —CF₂CH₂—;
X, Y, and Z are independently selected from the group consisting of hydrogen, N₃, halogen, C₁-C₆ alkyl, C₃-C₁₂ cycloalkyl, alkylamino, dialkylamino, C₁-C₆ alkynyl, substituted alkynyl, aryl, substituted aryl, —S-aryl, —S-substituted aryl, —O-aryl, —O-substituted aryl, NH-aryl, NH-substituted aryl, diarylamino, diheteroarylamino, arylalkyl, substituted arylalkyl, heteroaryl, substituted heteroaryl, —S-heteroaryl, —S-substituted heteroaryl, —O-heteroaryl, —O-substituted heteroaryl, —NH-heteroaryl, —NH-substituted heteroaryl, heteroarylalkyl, substituted heteroarylalkyl, heterocycloalkyl, and substituted heterocycloalkyl; or, in the alternative, X and Y or Y and Z taken together with the carbon atoms to which they are attached form an aryl, substituted aryl, heteroaryl, and substituted heteroaryl cyclic moiety;
j=0, 1,2,3,or 4;
m=0, 1, or 2;
s=0,l or 2;
R¹ is hydrogen, C₁-C₆ alkyl, C₃-C₁₂ cycloalkyl, substituted C₃-C₁₂ cycloalkyl, aryl, substituted aryl, arylalkyl, substituted arylalkyl, heteroaryl, substituted heteroaryl, heteroarylalkyl, substituted heteroarylalkyl, heterocycloalkyl, or substituted heterocycloalkyl;
R² is hydrogen, C₁-C₆ alkyl, C₃-C₁₂ cycloalkyl, substituted C₃-C₁₂ cycloalkyl, alkylamino, dialkyl amino, arylamino, diarylamino, aryl, substituted aryl, arylalkyl, substituted arylalkyl, heteroaryl, substituted heteroaryl, heteroarylalkyl, substituted heteroarylalkyl, heterocycloalkyl, or substituted heterocycloalkyl; and
R³ and R⁴ are each independently hydrogen or methyl.

9. A compound according to claim 8, wherein:
A is —(C═O)—O—R¹;
G is hydroxyl;
L is absent;
j=3;
m=s=1; and
R³ and R⁴ are hydrogen.

10. A compound according to claim 8, wherein:
A is —(C═O)—O-tert-butyl;
G is hydroxyl;
L is absent;
j=3;
m=s=1; and
R³ and R⁴ are hydrogen.

11. A pharmaceutical composition comprising an anti-hepatitis C virally effective amount of a compound according to claim 1 or 8, or a pharmaceutically acceptable salt, ester, or prodrug thereof, in combination with a pharmaceutically acceptable carrier or excipient.

12. A method of treating a hepatitis C viral infection in a mammal, comprising administering to the mammal an anti-hepatitis C virally effective amount of a pharmaceutical composition according to claim 11.

13. A method of inhibiting the replication of hepatitis C virus, the method comprising supplying a hepatitis C viral NS3 protease inhibitory amount of the pharmaceutical composition of claim 11.

14. The method of claim 12 further comprising administering concurrently an additional anti-hepatitis C virus agent.

15. The method of claim 14, wherein said additional anti-hepatitis C virus agent is selected from the group consisting of: α-interferon, β-interferon, ribavarin, and adamantine.

16. The method of claim 14, wherein said additional anti-hepatitis C virus agent is an inhibitor of another target in the hepatitis C virus life cycle, which is selected from the group consisting of: helicase, polymerase, metalloprotease, and IRES.
